(19) United States
(12) Patent Application Publication
(10) Pub. No.: US 2007/0259337 A1
(43) Pub. Date: Nov. 8, 2007

(54) Methods and systems for designing primers and probes

(75) Inventors: James Robert Hully, Salisbury, MA (US); Gilead Kedem, Medford, MA (US); Raymond P. Lauer, San Diego, CA (US)

Correspondence Address: DOCKETING SPECIALIST;SULLIVAN & WORCESTER LLP, ONE POST OFFICE SQUARE, BOSTON, MA, 02109, US

(73) Assignee: Intelligent Medical Devices, Inc., Cambridge, MA (US)

(21) Appl. No.: 11/605,942

(22) Filed: Nov. 29, 2006

Related U.S. Application Data
(60) Provisional application No. 60/740,582, filed on Nov. 29, 2005.

(51) Int. Cl. C12Q 1/70; C07H 21/00; C12P 19/34; G06F 17/30; G01N 33/48; C12Q 1/68

(52) U.S. Cl. 435/5; 435/6; 435/91.2; 536/23.1; 536/24.32; 536/24.33; 702/20; 707/104.1

(57) ABSTRACT

The invention provides methods for designing polynucleotide primers and probes that are optimized for hybridizing to a plurality of target nucleic acid variants by employing scoring and/or ranking steps that provide a positive or negative preference or “weight” to certain nucleotides in a candidate nucleic acid sequence. The particular scoring or ranking steps performed depend upon the intended use for the primer and/or probe, the particular target sequence, and the number of variants of that target sequence. The methods of the invention provide optimal primer and probe sequences because they hybridize to more target nucleic acid variants than primers and probes in the prior art.

### CROSS-REFERENCES TO RELATED APPLICATION

This application claims the benefit of U.S. Provisional Application No. 60/740,582, filed on Nov. 29, 2005, which is incorporated herein by reference.

### FIELD OF THE INVENTION

The invention relates to methods for designing nucleic acid primers and probes that are optimized for hybridizing to a plurality of target nucleic acid variants.

### BACKGROUND OF THE INVENTION

Current commercial software for selecting nucleic acid primers and probes identifies sequences based on their suitability for use in a nucleic acid amplification reaction such as polymerase chain reaction (PCR). Generally, the selection of a primer or a probe is determined by such parameters as sequence Tm, % GC content, sequential runs of certain bases, etc., and the software treats each nucleotide position of the target sequence as being equally important or representative.

This approach to primer and probe design has limited success if the target nucleic acid is genetically diverse. The genomes of many microorganisms, such as viruses and bacteria, show considerable intra-species variations. For example, there are at least 2000 different variants of human Influenza A listed in Genbank. Small changes in the nucleic acid sequence may represent the emergence of new and potentially more dangerous microorganisms. Similarly, these changes may alter the microbial proteins, thereby preventing their recognition by rapid antibody-based diagnostic tests. Such genetic variations within a single species can be a significant hurdle for those designing probes for diagnostic tests that use nucleic acid as a target.

To design a primer or probe for detecting nucleic acids having genetically diverse sequences, a multiple alignment of the target nucleic acid sequences is used to generate a consensus sequence. The consensus sequence is then assessed using primer and/or probe choosing software. Although existing software has some form of sequence annotation that restricts which region of the sequence can be used for selecting primers or probes, this is usually very limited and requires manual input. Furthermore, a primer or probe selected by this approach is only evaluated by its ability to perform PCR (i.e., how well it functions as primer or probe), and not on how many of the multiple target variants the primer or probe may bind to. Determining what percentage of target variants to which a particular candidate primer or probe may bind can be performed manually but is very time consuming, not reproducible, subject to error, and does not likely identify the optimal primer or probe sequence or set of primer or probe sequences.

A need therefore exists for a rapid, reproducible method for designing primers and probes that are useful in synthesizing, amplifying, and/or identifying genetically diverse target nucleic acids.

### SUMMARY OF THE INVENTION

The invention provides methods for designing polynucleotide primers and probes that are optimized for hybridizing to a plurality of target nucleic acid variants by employing scoring and/or ranking steps that provide a positive or negative preference or “weight” to certain nucleotides in a target nucleic acid variant sequence. The particular scoring or ranking steps performed depend upon the intended use for the primer and/or probe, the particular target nucleic acid sequence, and the number of variants of that target nucleic acid sequence. The methods of the invention provide optimal primer and probe sequences because they hybridize to more target nucleic acid variants than primers and probes in the prior art. The optimal primers and probes of the invention are useful, for example, for identifying and diagnosing the causative or contributing agents of a particular set of human disease symptoms. These agents can include infectious organisms (such as, for example, viruses, bacteria, fungi, and parasites), adjunct markers of infection (such as, for example, drug resistance 16s ribosomal RNA), and host factors (such as, for example, pharmacokinetic and inflammatory markers).

In one aspect, the invention provides methods for designing a primer for synthesizing (e.g., amplifying) a plurality of target nucleic acid variants by (a) identifying nucleotide identities between at least two target nucleic acid variant sequences that are representative of at least two target organisms or genes (e.g., pathogen or allelic variants); (b) selecting at least two candidate primer sequences that define a primer that can hybridize with the at least two target nucleic acid variant sequences; and (c) ranking the candidate primer sequences according to their percentage identity to the target nucleic acid variant sequences, or complements thereof, thereby determining an optimal candidate primer sequence for synthesizing a plurality of target nucleic acid variants. In another embodiment, the ranking step comprises ranking the primer(s) according to conservation score.

In another aspect, the invention provides methods for designing a probe for identifying a plurality of target nucleic acid variants by (a) identifying nucleotide identities between at least two target nucleic acid variant sequences that are representative of at least two target organism or gene variants (e.g., pathogen or allelic variants); (b) selecting at least two candidate probe sequences that define a probe that can hybridize with the at least two target nucleic acid variant sequences; and (c) ranking the candidate probe sequences according to their percentage identity to the target nucleic acid variant sequences, or complements thereof, thereby determining an optimal candidate probe sequence for identifying a plurality of target nucleic acid variants. In another embodiment, the ranking step comprises ranking the probe(s) according to conservation score.

The invention also provides methods for designing primer pairs for amplifying a plurality of target nucleic acid variants by (a) identifying nucleotide identities between at least two target nucleic acid variant sequences that are representative of at least two target organism or gene variants; (b) selecting at least two candidate forward primer sequences that define a forward primer that can hybridize with the at least two target nucleic acid variant sequences; (c) selecting at least two candidate reverse primer sequences that define a reverse primer that can hybridize with the at least two target nucleic acid variant sequences; (d) ranking the forward primer sequences according to their percentage identity to the target nucleic acid variant sequences, or complements thereof, thereby determining an optimal forward primer sequence for amplifying a plurality of target nucleic acid variants; and (e) ranking the reverse primer sequences according to their percentage identity to the target nucleic acid variant sequences, or complements thereof, thereby determining an optimal reverse primer sequence for amplifying a plurality of target nucleic acid variants.

In another embodiment, the invention provides methods for designing sets of primer pairs for amplifying a plurality of target nucleic acid variants and a probe for detecting an amplicon generated by the amplification. The methods comprise the additional step of (f) selecting at least two candidate probe sequences that define a probe that can hybridize with the at least two target nucleic acid variant sequences and (g) ranking the probe sequences according to their percentage identity to the target nucleic acid variant sequences, or complements thereof, thereby determining an optimal probe sequence for identifying a plurality of target nucleic acid variants.

The scoring or ranking steps that are used in the methods of the invention include, for example, at least one step of (i) determining a target sequence score for the target nucleic acid sequence(s); (ii) determining a mean conservation score for the target nucleic acid sequence(s); (iii) determining a mean coverage score for the target nucleic acid sequence(s); (iv) determining 100% conservation score of a portion (e.g., 5′ end, center, 3′ end) of the target nucleic acid sequence(s); (v) determining a species score (vi) determining a strain score; (vii) determining a subtype score; (viii) determining a serotype score; (ix) determining an associated disease score; (x) determining a year score; (xi) determining a country of origin score; (xii) determining a duplicate score; (xiii) determining a patent score; and (xiv) determining a minimum qualifying score. These scores represent steps in determining nucleotide or whole target nucleic acid sequence preference, while tailoring the primer and/or probe sequences so that they hybridize to a plurality of target nucleic acid variants. The methods of the invention also may comprise the step of allowing for one or more nucleotide changes when determining identity between the candidate primer and probe sequences and the target nucleic acid variant sequences, or their complements.

In another embodiment, the methods of the invention comprise the step of comparing the candidate primer and/or probe nucleic acid sequences to exclusion nucleic acid sequences and rejecting those candidate nucleic acid sequences if they share identity with the exclusion nucleic acid sequences.

In another embodiment, the methods of the invention comprise the step of comparing the candidate primer and/or probe nucleic acid sequences to inclusion nucleic acid sequences and rejecting those candidate nucleic acid sequences if they do not share identity with the inclusion nucleic acid sequences.

In an embodiment, the target nucleic acid sequence is a disease marker, such as a pathogen nucleic acid, for example Influenza A matrix protein gene (INFA-MP); Influenza B non-structural protein gene (INFB-NS); Respiratory Syncytial Virus A Glycoprotein gene (RSVA-G); Respiratory Syncytial Virus B Glycoprotein gene (RSVB-G); Respiratory Syncytial Virus A Nucleocapsid gene (RSVA-N); Respiratory Syncytial Virus B Nucleocapsid gene (RSVB-N); Parainfluenza 1 HN gene (PIV1-HN); Parainfluenza 2 HN gene (PIV2-HN); Parainfluenza 3 HN gene (PIV3-HN); Adenovirus-B Hexon gene (ADVB-H); Adenovirus-C Hexon gene (ADVC-H); Adenovirus-E Hexon gene (ADVE-H), the ribosomal RNA subunits of fastidious & respiratory bacteria such as Mycoplasma pneumoniae, Chlamydia pneumoniae, Chlamydia psittaci, Legionella pneumophila, Mycobacterium tuberculosis, Bordetella pertussis, Pneumocystis carinii, Streptococcus pneumoniae, Haemophilus influenzae, Staphlococcus aureus, Pseudomonas aeruginosa, Klebsiella pneumoniae, Acinetobacter baumannii, & Moraxella catarrhalis; for pathogens associated with perinatal diseases, these would include the glycoprotein D (gD), glycoprotein G (gG), & DNA polymerase genes of human Herpes simplex virus 1 & 2, streptococcal C5a peptidase gene of Streptococcus agalactiae (Group B Strep), the DNA gyrase subunit A (gyrA), glutamine synthatase (glnA), outer membrane porin protein (porA), Neisseria surface protein A (nspA) for Neisseria gonorrhoeae, and the major outer membrane protein A (ompA) for Chlamydia trachomatis.

In another embodiment, the target nucleic acid is a genetic marker, such as, for example, of microbial drug resistance (β Lactamases, mecA/PBP2a gene, Vancomycin resistance −vanA & vanB, Rifampin resistance, Isoniazid resistance), human markers of pharmacogenomics, inflammation, infection (such as an acute phase reactant nucleic acid or inflammation associated nucleic acid), allergy, neoplasia (e.g., genes associated with disease susceptibility such as p53 and BRAC1), autoimmunity, immunodeficiency, chronic obstructive pulmonary disease (COPD), and jaundice. The target nucleic acid may be any disease-related nucleic acid, for example a nucleic acid that is representative of an infectious agent or microbe, e.g., a virus, a bacteria, a fungus, a parasite, a mycoplasma, a rickettsia, a chlamydia, a protozoa, and a plant cell (such as an algae or pollen). The target nucleic acid may also be a specific genetic sequence indicative of a genetic disorder of a subject being tested. For example, a genetic disorder can be marked by a mutation of a gene, a single nucleotide polymorphism (SNP), an extra copy of a normal chromosome or gene, or a missing gene. A target can also be a marker for a therapeutic optimization factor, such as a microbial gene that provides resistance, tolerance, or susceptibility to a particular drug. Such a therapy optimization factor can also be a genetic feature of the subject that makes the subject resistant, tolerant, or intolerant (e.g., allergic) to a particular drug.

In many autoimmune diseases, there is association of particular HLA antigens in populations of individuals with certain diseases. Primers and probes are designed to detect HLAs such as: HLA B27; HLA B38; HLA DR8; HLA DR5; HLA Dw4/DR4; HLA Dw3; 7HLA DR3; HLA DR4; HLA B5; HLA Cw6; HLA A26; HLA B51; HLA B8; HLA Dw3; HLA B35; HLA DR2; HLA B12; and HLA A3. The methods and nucleic acids of the invention can be used to detect gene mutations that affect the autoimmune syndrome, such as: Fas; FasL; and the Canale-Smith syndrome, including deficiencies of early and late complement components associated with autoimmune diseases. Mutations in the following genes are associated with complement deficiencies and/or autoimmune syndrome: C1 (C1q, C1r, C1s); C4; C2; C1 inhibitor; C3; D; Properdin; I; P; C5, C6, C7, C8, and C9. In addition, mutations/allelic variations that result in immunodeficiency include: A) SCID associated with defective cytokine signaling—gammac; Jak3; IL-2; IL-2Ra; and IL-7Ra; B) SCID associated with TCR related defects—CD3g; CD3e; and ZAP70; C) HLA class II deficiency—CIITA; RFX5; and RFXB; D) HLA class I deficiency (bare leukocyte syndrome)—TAP1 and TAP2; E) Immunodeficiency associated with defects in enzymes other than kinases—ADA deficiency and PNP deficiency; F) X-linked hyper—IgM-CD40 ligand; G) X-linked agammaglobulinemia (Bruton)—Btk; H) Non-X-linked agammaglobulinemia-m heavy chain; I) Wiskot-Aldrich Syndrome—WASP; J) Ataxia telangiectasia—ATM; K) DiGeorge anomaly—21q; L) Autoimmune lymphoproliferative syndrome—Fas; M) XLP-SH2D1A/SAP; N) TRAPS—TNFRSF1A; and/or O) Susceptibility to microbacterial infections—IFN-gammaR1; IFN-gammaR2; IL-12p40.

The target nucleic acid may share homology, similarity, or identity with nucleic acids in at least two groups such as two different kingdoms, phyla, classes, orders, families, genera, species, subtypes, and genotypes, for example. In another embodiment, the target comprises a number of serotypes or phenotypes. The primers and probes of the invention are capable of hybridizing to at least two members of the above groups or a combination thereof, and preferably a plurality thereof.

In an embodiment, the step of identifying target nucleic acid variant identities in the methods of the invention involves aligning the target nucleic acid variant sequences. A manual alignment of target nucleic acid variant sequences against sequences from a database (e.g., public and annotated) may be performed, for example. The databases used in an embodiment of the methods of the invention include annotated databases, such as the PriMD™ database described herein. Alternatively, the database could be any of a number of nucleic acid databases, such as, for example, the Influenza Sequence Database, the Ribosomal Database project, STD database, and/or Genbank database. Alternatively, the alignment is performed using a program such as, for example, BLAST, ClustalW, ClustalX, PileUp (GCG), MULTALIGN, DNAStar's Lasergene, and Tcoffee. In an embodiment, the alignment is performed using a sum of pairs scoring method and/or optimization using an evolutionary tree. The identifying step of the methods of the invention may further comprise editing the alignment by removing at least one 5′ nucleotide and/or at least one 3′ nucleotide from at least one nucleic acid sequence if the sequence does not fit into the alignment. The alignment may also be repeated after the editing step.

In an embodiment of the methods of the invention, the selecting step (b) comprises using a polymerase chain reaction (PCR) penalty score formula comprising at least one of a weighted sum of: primer Tm−optimal Tm; difference between primer Tms; amplicon length−minimum amplicon length; and distance between the primer and a TaqMan probe.

In an embodiment, the selecting step comprises determining the ability of the candidate sequence to hybridize with the most target nucleic acid variant sequences (e.g., the most target organisms or genes). In another embodiment, the selecting step comprises determining which sequences have mean conservation scores closest to 1, wherein a standard of deviation on the mean conservation scores is also compared.

In other embodiments, the methods further comprise the step of evaluating which infectious agent target nucleic acid variant sequences are hybridized by an optimal forward primer and an optimal reverse primer, for example, by determining the number of base differences between target nucleic acid variant sequences in a database. For example, the evaluating step may comprise performing an in silico polymerase chain reaction, involving (1) rejecting the forward primer and/or reverse primer if it does not meet inclusion or exclusion criteria; (2) rejecting the forward primer and/or reverse primer if it does not amplify a medically valuable nucleic acid; (3) conducting a BLAST analysis to identify forward primer sequences and/or reverse primer sequences that overlap with a published and/or patented sequence; (4) and/or determining the secondary structure of the forward primer, reverse primer, and/or target. In an embodiment, the evaluating step includes evaluating whether the forward primer sequence, reverse primer sequence, and/or probe sequence hybridizes to sequences in the database other than the nucleic acid sequences that are representative of the target variants.

In another aspect, the invention provides a software program that automates the design steps of the invention. Such a program, designated herein as the PriMD™ software, may be part of an integrated PriMD™ system that also includes a database called the PriMD™ database. The database of the invention stores the information both used in and derived from the methods of the invention for future use.

In another aspect, the invention provides primer and probe nucleic acids as well as amplicon nucleic acids generated by the amplification of target nucleic acid variants by the primers.

In an embodiment, the invention provides nucleic acids (e.g., oligonucleotides and polynucleotides) comprising a sequence that shares at least about 60-70% identity with the sequence of any one of SEQ ID NOs: 1-94, or the complement thereof. In another embodiment, the invention provides a nucleic acid comprising a sequence that shares at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity with the sequence of any one of SEQ ID NOs: 1-94, or complement thereof. The probe and/or primer nucleic acid sequences of the invention are optimal for identifying numerous variants of a target nucleic acid, e.g., from a target pathogen. In an embodiment, the nucleic acids of the invention are primers for the synthesis (e.g., amplification) of target nucleic acid variants and/or probes for identification, isolation, detection, or analysis of target nucleic acid variants, e.g., an amplified target nucleic acid variant that is amplified using the primers of the invention.

Target pathogens include, but are not limited to, Acanthamoeba family; Ascaris family (including Ascaris lumbricoides); Acetobacter family (including Acetobacter aurantius); Actinobacillus family (including Actinobacillus actinomycetemcomitans); Actinomyces family; Adenovirus family (including Mastadenoviruses, Aviadenoviruses, Atadenoviruses, and Siadenoviruses); Aeromonas family; Agrobacterium family (including Agrobacterium tumefaciens); Ancylostoma family (including Ancylostoma duodenal); Arcanobacterium family (including Arcanobacterium haemolyticum); Arenavirus family (including Ippy virus, Lassa virus, Lymphocytic choriomeningitis virus, and Mobala virus); Ascaris family (including Ascaris lumbricoides); Astrovirus family (including Avastrovirus and Mamastrovirus); Azorhizobium family (including Azorhizobium caulinodans); Azotobacter family (including Azotobacter vinelandii); Bacillus family (including Bacillus anthracis, Bacillus brevis, Bacillus cereus, Bacillus fusiformis, Bacillus licheniformis, Bacillus megaterium, Bacillus stearothermophilus, and Bacillus subtilis); Bacteroides family (including Bacteroides fragillis, Bacteroides gingivalis, and Bacteroides melaminogenicus); Balantidium family (including Balantidium coli); Bartonella family (including Bartonella henselae, and Bartonella quintana); Blastocystic family (including Blastocystic hominis); Blastomyces family (including Blastomyces dermatitidis); Bordetella family (including Bordetella pertussis, and Bordetella bronchiseptica); Borellia family (including Borellia burgdorferi); Brucella family (including family abortus, Brucella melitensis, and Brucella suis); Brugia family (including Brugia malayi and Brugia timori); Bunyavirus family (including Phleboviruses, Nairoviruses, Hantaviruses, and Tospoviruses); Burkholderia family (including Burkholderia pseudomallei, and Burkholderia pseudomallei); Calcivirus family (including Norwalk virus and Hepatitis E); Calaymmatobacterium family (including Calaymmatobacterium granulomatis); Campylobacter family (including Campylobacter coli, Campylobacter jejuni, and Campylobacter pylori); Candida family (including Candida albicans); Chlamydiae family (including Chlamydia pneumoniae, Chlamydia psittaci, and Chlamydia trachomatis); Chlamydophila family (including Chlamydophila pneumoniae, and Chlamydophila psittaci); Clonorchis family (including Clonorchis sinensis); Clostridium family (including Clostridium botulinum, Clostridium tetani, Clostridium welchii, Clostridium difficile, and Clostridium perfringens; Coccidioides family (including Coccidioides immitis); Coronavirus family (including coronaviruses and toroviruses); Corynebacterium family (including Corynebacterium diphtheriae, Corynebacterium fusiforme, and Corynebacterium ulcerans); Coxiella family (including Coxiella burnetii); Cryptococcus family (including Cryptococcus neoformans); Cryptosporidium family; Deltavirus family (including Hepatitis D); Diphyllobothrium family (including Diphyllobothrium latum); Echovirus family; Ehrlichia family (including Ehrlichia chaffeensis); Entamoeba family (including Entamoeba histolytica); Enterobius family (including Enterobius vermicularis); Enterococcus family (including Enterococcus avium, Enterococcus durans, Enterococcus faecalis, Enterococcus faecium, Enterococcus galllinarum, and Enterococcus maloratus); Escherichia family (including Escherichia coli); Eurotiaceae family (including Aspergillus flavus, Aspergillus fumigatus, Aspergillus niger, Aspergillus nidulans, and Aspergillus terreus); Fasciola family (including Fasciola hepatica); Fasciolopsis family (including Fasciolopsis buski); Filovirus family (including Ebola virus); Flavivirus family (including the group B arboviruses, Hepatitis C, and Dengue); Francisella family (including Francisella tularensis); Fusobacterium family (including nucleatum); Gardnerella family (including Gardnerella vaginalis); Giardia family (including Giardia lamblia); Gymnoascaceae family (including Histoplasma capsulatum); Haemophilus family (including Haemophilus influenzae, Haemophilus ducreyi, Haemophilus parainfluenzae, Haemophilus pertussis, and Haemophilus vaginalis); Helicobacter family (including Helicobacter pylori); Hepadna virus family (includes Hepatitis B); Herpes virus family (including Alphaherpesviruses, Betaherpesviruses, and Gammaherpesviruses); Hymenolepis family (including Hymenolepis nana); Isospora family (including Isospora belli); Klebsiella family (including Klebsiella pneumoniae); Lactobacillus family (including Lactobacillus acidophilus, and Lactobacillus casei); Legionella family (including Legionella pneumophila); Leishmania family (including Leishmania donovani); Leptospira family; Listeria family (including Listeria monocytogenes); Methanobacterium family (including Methanobacterium extroquens); Microbacterium family (including Microbacterium multiforme); Micrococcus family (including Micrococcus luteus); Moraxella family (including Moraxella catarrhalis); Mycobacterium family (including Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, and Mycobacterium tuberculosis); Mycoplasma family (including Mycoplasma fermentans, Mycoplasma genitalium, Mycoplasma hominis, and Mycoplasma pneumoniae); Naegleria family; Necator family (including Necator americanus); Neisseria family (including Neisseria gonorrhoeae, and Neisseria meningitidis); Nocardia family (including Nocardia asteroides); Onchocerca family (including Onchocerca volvulus); Orthomyxovirus family (includes human & avian Influenza viruses types A, B and C); Paracoccidioides family (including Paracoccidioides brasiliensis); Paramyxovirus family (including the Paramyxoviruses, Rubulaviruses, Morbilliviruses and Pneumoviruses); Papova virus family (includes Human Papilloma virus, JC Virus, and BK virus); Paracoccidioides family (includes Paracoccidioides brasiliensis); Paragonimus family (including Paragonimus westermani); Parvovirus family (includes Densoviruses & Parvoviruses); Pasteurella family (includes Pasteurella multocida, and Pasteurella tularensis); Peptostreptococcus family (including Peptostreptococcus magnus, Peptostreptococcus prevotii, and Peptostreptococcus anaerobius); Picorna virus family (including Enteroviruses, Rhinoviruses, and Hepatoviruses); Pityrosporum family (including Pityrosporum folliculitis); Plasmodium family; Pneumocystis family (including Pneumocystis carinii); Poxvirus family (including smallpox and molluscum contagiosum virus); Porphyromonas family (including Porphyromonas gingivalis); Prevotella family (including Prevotella melaminogenica); Proteus family (including Proteus mirabilis); Pseudomonas family (including Pseudomonas aeruginosa, and Pseudomonas maltophilia); Reovirus family (including Orbiviruses and Rotaviruses); Retrovirus family (includes Alpharetroviruses, Betaretroviruses, Gammaretroviruses, Deltaretroviruses, Epsilonretroviruses, Lentiviruses and Spumaviruses); Rhabdovirus family (including vesiculoviruses, lyssaviruses, ephemeroviruses, norvirhabdoviruses, cytorhabdoviruses, and nucleorabdoviruses); Rhizobium family (including Rhizobium radiobacter); Rickettsiae family (including Rickettsia rickettsia, Rickettsia conorii, Rickettsia prowazekii, Rickettsia quintana, Rickettsia trachoma, Rickettsia typhi, and Rickettsia tsutsugamushi); Rochalimaea family (including Rochalimaea henselae, and Rochalimaea quintana); Rothia family (including Rothia dentocariosa); Salmonella family (including Salmonella enteritidis, Salmonella typhi, and Salmonella typhimurium; SARS-like virus family; Schistosoma family (including Schistosoma haematobium, Schistosoma mansoni and Schistosoma japonicum); Septata family (including Septata intestinalis); Serratia family (including Serratia marcescens); Shigella family (including Shigella dysenteriae); Spirillum family (including Spirillum minus); Spirochaeta family; Sporothrix family (including Sporothrix schenckii); Staphylococcus family (including Staphylococcus aureus, and Staphylococcus epidermidis); Streptococcus family (including Streptococcus agalactiae, Streptococcus equi, Streptococcus equisimilis, Streptococcus zooepidemicus, Streptococcus pneumoniae, Streptococcus pyogenes, Streptococcus avium, Streptococcus bovis, Streptococcus cricetus, Streptococcus faceium, Streptococcus faecalis, Streptococcus ferus—Streptococcus gallinarum, Streptococcus lactis, Streptococcus mitior, Streptococcus mitis, Streptococcus mutans, Streptococcus oralis, Streptococcus rattus, Streptococcus salivarius, Streptococcus sanguis, and Streptococcus sobrinus); Taenia family (including Taenia saginata and Taenia solium); Tinea family (including Tinea versicolor); Togovirus family (including Alphaviruses—encephalitis viruses, and Rubiviruses—Rubella and German measles); Toxocara family (including Toxocara canis); Toxoplasma family (including Toxoplasma gondii); Treponema family (including Treponema pallidum); Trichinella family (including Trichinella spiralis); Trichomonas family (including Trichomonas vaginalis); Trichuris family (including Trichuris trichiuria); Trypanosoma family (including Trypanosoma brucei and Trypanosoma cruzi); Ureaplasma family (including Ureaplasma urealyticum); Vibrio family (including Vibrio cholerae, Vibrio comma, Vibrio vulnificus, and Vibrio parahaemolyticus); Wuchereria family (including Wuchereria bancrofti); Xanthomonas family (including Xanthomonas maltophilia); Yersinia family (including Yersinia enterocolitica, Yersinia pestis, and Yersinia pseudotuberculosis); Zygomycetes family (including Absidia corymbifera, Rhizomucor pusillus, and Rhizopus arrhizus).

In an embodiment, the nucleic acids of the invention hybridize with at least N different target nucleic acid variants, wherein N is any integer from 1 to the total number of known variants of a target nucleic acid. N, therefore, may vary over time for a given target nucleic acid (e.g., if new variants are discovered). Because the methods of the invention provide for the identification of optimal primers and probes, and sets thereof, and combinations of sets thereof, that can hybridize with a larger number of target variants than available primers and probes, N is higher for the primers and probes of the invention than it is for currently used commercial primers and probes.

In another embodiment, the invention provides nucleic acids that comprise and/or hybridize to a nucleic acid comprising the sequence of any one of SEQ ID NOS 1-71, or the complement thereof. In an embodiment, the nucleic acid hybridizes to the target nucleic acid under low stringency hybridization conditions. In another embodiment, the nucleic acid hybridizes to the target nucleic acid under high stringency hybridization conditions.

In another embodiment, the invention provides nucleic acids that comprise and/or hybridize to a nucleic acid comprising the sequence of SEQ ID NOs: 49-71 or the complement thereof. These regions were identified as having a high level of conservation and are the regions in the target nucleic acid variants from which candidate primers and probes are derived.

In another embodiment, the invention provides nucleic acids that comprise and/or hybridize to the conserved nucleotides of the consensus sequences of any one of SEQ ID NOs: 72-94 (FIG. 6), or the complements thereof. In an embodiment, these nucleic acids of the invention are able to hybridize with a target nucleic acid of the invention, or complement thereof.

In other aspects, the invention also provides vectors (e.g., plasmid, phage, expression), cell lines (e.g., mammalian, insect, yeast, bacterial), and kits comprising any of the sequences of the invention described herein. The invention further provides target nucleic acid variant sequences that are identified, for example, using the methods of the invention. In an embodiment, the target nucleic acid variant sequence is an amplification product. In another embodiment, the target nucleic acid variant sequence is a native or synthetic nucleic acid. The primers, probes, and target nucleic acid variant sequences, vectors, cell lines, and kits may have any number of uses, such as diagnostic, investigative, confirmatory, monitoring, predictive or prognostic.

A wide variety of human diagnostic kits can be created using the methods and nucleic acids described herein. These kits provide information to a clinician or physician about the causes for specific symptoms, or clusters of symptoms, presented by a patient. Specific examples of human diagnostic kits include: Headache/fever/meningismus (Meningitis) Kit, Cough/fever/chest discomfort/dyspnea (Pneumonia) Kit, Jaundice (Liver failure) Kit, Recurrent Infection (Immunodeficiency) Kit, Joint Pain Kit, and many others.

Human detection kits provide information about the current state of a patient's condition, such as the patient's immunization or immunocompetence state or the presence of a disease in the body (e.g., a disease not yet showing symptoms), or the condition of a medical product, such as a blood supply or a donated organ.

Animal diagnostic and screening kits allow comprehensive, cost-effective, and rapid diagnosis of numerous congenital and acquired diseases based on an animal's clinical presentation of specific symptoms. In addition, animal exposure to different pathogens or pathogen products (e.g., toxins) can be evaluated, as well as specific genes and/or diseases linked to improved breeding (e.g., the size of the litter, and meat/milk production). In an embodiment, these kits are species-specific. Examples include: Laboratory Mouse Kit, Sheep Kit, Laboratory Rat Kit, Dog Kit, Simian Kit, Racing Horse Kit, Cattle Kit, Chicken Kit, Porcine Kit, Lamb Kit, Fish Kit.

Agriculture Kits allow comprehensive, cost-effective, and rapid diagnosis of numerous congenital and acquired diseases based on plant's clinical presentation of specific symptoms. In addition, plant exposure to different pathogens is evaluated, as well as specific genes and/or diseases linked to improved plant growth (e.g., the size of the plant, the corn/rice production, etc.). In an embodiment, these kits are species-specific. Examples include: Corn Kit, Cotton Kit, Tobacco Kit, and Rice Kit.

The invention covers additional, more specific kits as follows: forensic kits; food-borne pathogens (e.g., viral and microbial) and antibiotic resistance kit; inspection of imported goods—agricultural and livestock kit; pesticide kit; inspection of cosmetics (e.g., mad cow disease) kit; bioterrorism kit (e.g., smallpox, anthrax, plague, botulism, tularemia, and hazardous chemical agents); and influenza surveillance kit (e.g., that screens all known strains of influenza).

In an embodiment, the probes of the invention comprise a label, such as a fluorescent label, a chemiluminescent label, a radioactive label, biotin, gold, dendrimers, aptamer, enzymes, proteins, and molecular motors. In an embodiment, the probe is a hydrolysis probe, such as, for example, a TaqMan probe. In other embodiments, the probes of the invention are molecular beacons, SYBR Green primers, or fluorescence energy transfer (FRET) probes.

In an embodiment, the nucleic acids of the invention are attached to a solid support, such as, for example, a microarray, multiwell plate, column, bead, glass slide, polymeric membrane, glass microfiber, plastic tubes, cellulose, and carbon nanostructures.

In another embodiment, the invention provides primer pairs for amplifying target nucleic acid variants. In an embodiment, the primer pair comprises a forward (e.g., first) primer and a reverse (e.g., second) primer. For example, forward primers are defined by the sequences that share at least about 70% identity with at least one of the sequences of SEQ ID NOs: 1, 5, 9, 13, 17, 21, 25, 29, 33, 37, 41, 45, 73, 76, 80, 82, 85, 88, 91, and 93, or the complement thereof. Reverse primers are defined by the sequences that share at least about 70% identity with at least one of the sequences of SEQ ID NOs: 3, 7, 11, 15, 19, 23, 27, 31 35, 39, 43, 47, 74, 77, 79, 83, 86, 89, 92, 95, 98, and 101, or the complement thereof. In an embodiment, the primer pair amplifies at least N different target nucleic acid variants, wherein N comprises at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the known variants for a particular target nucleic acid sequence.

In another embodiment, the forward primers hybridize to a nucleic acid comprising at least one of the sequences of SEQ ID NOs: 1, 5, 9, 13, 17, 21, 25, 29, 33, 37, 41, 45, 73, 76, 79, 82, 85, 88, 91, 94, 97, and 100, or complement thereof, and reverse primers hybridize to a nucleic acid comprising at least one of the sequences of SEQ ID NOs: 3, 7, 11, 15, 19, 23, 27, 31, 35, 39, 43, 47, 74, 77, 80, 83, 86, 89, 92, 95, 98, and 101, or complement thereof. In an embodiment, the primer hybridizes to the nucleic acid under low stringency hybridization conditions. In another embodiment, the primer hybridizes to the nucleic acid under high stringency hybridization conditions. In an embodiment, the primer pair amplifies at least N different target nucleic acid variants, wherein N comprises at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the know variants for a particular target nucleic acid sequence.

In another embodiment, the forward primer comprises the sequence CAAGA, wherein the oligonucleotide hybridizes to an INFA-MP nucleic acid comprising the sequence of SEQ ID NO: 49, or the complement thereof.

In another embodiment, the forward primer comprises the sequence ATAGA, wherein the oligonucleotide hybridizes to an INFB-NS nucleic acid comprising the sequence of SEQ ID NO: 51, or the complement thereof.

In another embodiment, the forward primer comprises the sequence AAACA, wherein the oligonucleotide hybridizes to an RSVA-G nucleic acid comprising the sequence of SEQ ID NO: 52, or the complement thereof.

In another embodiment, the forward primer comprises the sequence TCATC, wherein the oligonucleotide hybridizes to an RSVB-G nucleic acid comprising the sequence of SEQ ID NO: 54, or the complement thereof.

In another embodiment, the forward primer comprises the sequence ATCTT, wherein the oligonucleotide hybridizes to an RSVA-N nucleic acid comprising the sequence of SEQ ID NO: 56, or the complement thereof.

In another embodiment, the forward primer comprises the sequence AGGAT, wherein the oligonucleotide hybridizes to an RSVB-N nucleic acid comprising the sequence of SEQ ID NO: 57, or the complement thereof.

In another embodiment, the forward primer comprises the sequence ACTCA, wherein the oligonucleotide hybridizes to an PIV1-HN nucleic acid comprising the sequence of SEQ ID NO: 59, or the complement thereof.

In another embodiment, the forward primer comprises the sequence TTCTC, wherein the oligonucleotide hybridizes to an PIV2-HN nucleic acid comprising the sequence of SEQ ID NO: 61, or the complement thereof.

In another embodiment, the forward primer comprises the sequence CTATC, wherein the oligonucleotide hybridizes to an PIV3-HN nucleic acid comprising the sequence of SEQ ID NO: 64, or the complement thereof.

In another embodiment, the forward primer comprises the sequence AGATG, wherein the oligonucleotide hybridizes to an ADVB-H nucleic acid comprising the sequence of SEQ ID NO: 67, or the complement thereof.

In another embodiment, the forward primer comprises the sequence CTCGG, wherein the oligonucleotide hybridizes to an ADVC-H nucleic acid comprising the sequence of SEQ ID NO: 69, or the complement thereof.

In another embodiment, the forward primer comprises the sequence GAACT, wherein the oligonucleotide hybridizes to an ADVE-H nucleic acid comprising the sequence of SEQ ID NO: 71, or the complement thereof.

In another embodiment, the reverse primer comprises the sequence GGACT, wherein the oligonucleotide hybridizes to an INFA-MP nucleic acid comprising the sequence of SEQ ID NO: 50, or the complement thereof.

In another embodiment, the reverse primer comprises the sequence TGTAA, wherein the oligonucleotide hybridizes to an INFB-NS nucleic acid comprising the sequence of SEQ ID NO: 51, or the complement thereof.

In another embodiment, the reverse primer comprises the sequence CTGCA, wherein the oligonucleotide hybridizes to an RSVA-G nucleic acid comprising the sequence of SEQ ID NO: 53, or the complement thereof.

In another embodiment, the reverse primer comprises the sequence TTAGC, wherein the oligonucleotide hybridizes to an RSVB-G nucleic acid comprising the sequence of SEQ ID NO: 55, or the complement thereof.

In another embodiment, the reverse primer comprises the sequence TAAAC, wherein the oligonucleotide hybridizes to an RSVA-N nucleic acid comprising the sequence of SEQ ID NO: 56, or the complement thereof.

In another embodiment, the reverse primer comprises the sequence GGAGT, wherein the oligonucleotide hybridizes to an RSVB-N nucleic acid comprising the sequence of SEQ ID NO: 58, or the complement thereof.

In another embodiment, the reverse primer comprises the sequence TGCTT, wherein the oligonucleotide hybridizes to an PIV1-HN nucleic acid comprising the sequence of SEQ ID NO: 60, or the complement thereof.

In another embodiment, the reverse primer comprises the sequence TCATC, wherein the oligonucleotide hybridizes to an PIV2-HN nucleic acid comprising the sequence of SEQ ID NO: 63, or the complement thereof.

In another embodiment, the reverse primer comprises the sequence ATAAC, wherein the oligonucleotide hybridizes to an PIV3-HN nucleic acid comprising the sequence of SEQ ID NO: 66, or the complement thereof.

In another embodiment, the reverse primer comprises the sequence TAATT, wherein the oligonucleotide hybridizes to an ADVB-H nucleic acid comprising the sequence of SEQ ID NO: 68, or the complement thereof.

In another embodiment, the reverse primer comprises the sequence TTCAG, wherein the oligonucleotide hybridizes to an ADVC-H nucleic acid comprising the sequence of SEQ ID NO: 70, or the complement thereof.

In another embodiment, the reverse primer comprises the sequence GATGT, wherein the oligonucleotide hybridizes to an ADVE-H nucleic acid comprising the sequence of SEQ ID NO: 71, or the complement thereof.

In another aspect the invention provides methods for amplifying a plurality of target nucleic acid variants by amplifying at least a portion of a target nucleic acid variant in a sample using a primer pair of the invention. The invention also provides methods for determining the presence or absence of a target nucleic acid variant in a sample by detecting the presence or absence of a native target nucleic acid variant sequence (e.g., RNA or DNA), a cDNA copy of a native target nucleic acid variant sequence, or an amplification product. In an embodiment, detection of the amplification product of the primer pair and the target native nucleic acid variant is indicative of the presence of the native target variant in the sample.

The sample may be a tissues sample, such as, for example, blood, serum, plasma, sputum, urine, stool, skin, cerebrospinal fluid, saliva, gastric secretions, and tear fluid. In an embodiment, the sample is obtained by an oropharyngeal swab, nasopharyngeal swab, throat swab, nasal aspirate, nasal wash, or fluid collected from the ear, eye, mouth, or respiratory airway. The tissue sample may be fresh, fixed, preserved, or frozen.

The target nucleic acid variant that is amplified may be RNA or DNA or a modification thereof. In an embodiment, the amplifying step comprises isothermal or non-isothermal reaction such as polymerase chain reaction, Scorpion™ primers, Molecular Beacons, SimpleProbes, HyBeacons, Cycling Probe Technology, Invader Assay, Self-sustained Sequence Replication, Nucleic Acid Sequence-based Amplification, Ramification Amplifying Method, Hybridization Signal Amplification Method, Rolling Circle Amplification, Multiple Displacement Amplification, Thermophilic Strand Displacement Amplification, Transcription-mediated Amplification, Ligase Chain Reaction, Signal Mediated Amplification of RNA Technology, Split Promoter Amplification Reaction, Ligase Chain Reaction, Q-Beta Replicase, Isothermal Chain Reaction, One Cut Event Amplification System, Loop-mediated Isothermal Amplification, Molecular Inversion Probes, Ampliprobe, Headloop DNA amplification, and Ligation Activated Transcription. In an embodiment, the amplifying step is conducted on a solid support, such as a multiwell plate, array, column, bead, glass slide, polymeric membrane, glass microfiber, plastic tubes, cellulose, and carbon nanostructures. In an embodiment, the amplifying step comprises in situ hybridization. The detecting step may comprise gel electrophoresis, fluorescence resonant energy transfer, or hybridization to a labeled probe, such as a probe labeled with biotin, at least one fluorescent moiety, an antigen, a molecular weight tag, and a modifier of probe Tm. In an embodiment, the detecting step comprises measuring fluorescence, mass, charge, and/or chemiluminescence.

In another aspect, the present invention provides methods for identifying a compound capable of modulating the expression of a target nucleic acid variant in a cell. The methods comprise (i) incubating a cell with a test compound under conditions that permit the compound to exert a detectable regulatory influence over a target nucleic acid variant gene, thereby altering the target nucleic acid variant gene expression; and (ii) detecting an alteration in the target nucleic acid variant gene expression.

In another embodiment, the present invention provides methods for diagnosing the presence of, or a predisposition to the development of, a disorder associated with abnormal target nucleic acid variant gene DNA levels, abnormal target nucleic acid variant gene RNA levels, or abnormal target nucleic acid variant gene activity. The present invention also provides methods for establishing target nucleic acid variant gene expression profiles for diseases or disorders, and methods for diagnosing and treating a disease or disorder using such expression profiles. In yet another embodiment, the invention provides methods for identifying an organism (e.g., of food, environmental, beverage, or veterinary origin), methods for determining a prognosis, methods for monitoring a drug therapy, methods for quantifying or qualifying virulence, drug resistance, or the presence of a bioterror threat.

According to yet another embodiment, a computer-implemented system for identifying oligonucleotides for detecting multiple variants of a target includes a user interface for specifying a target. The system further includes software for reading a multiple alignment of nucleic acid sequences for a plurality of variants of the target and software for generating a candidate sequence based at least in part upon the multiple alignment. The system still further includes software for computing the sequences of a plurality of oligonucleotides that are complementary to portions of the candidate sequence and software for assigning a quality metric to each computed oligonucleotide responsive to an extent to which the respective oligonucleotide aligns with each of the variants of the target.

According to a further embodiment, a computer-implemented system is provided for identifying oligonucleotide sets for detecting target nucleic acid variants. The system includes a user interface for specifying a target and a data collection for storing a plurality of data. The data collection includes nucleic acid sequences for a plurality of known targets, oligonucleotide sets corresponding to the nucleic acid sequences, or complements thereof, and additional data, comprising at least one of alignment data, demographic data, patent data, and commercial data. The system further includes software for identifying any oligonucleotide sets in the data collection that are candidates for detecting the specified target nucleic acid and software for computing at least one quality metric for each identified oligonucleotide set responsive to any of the additional data stored in the data collection.

According to another embodiment, a computer-implemented system is provided for identifying oligonucleotide sets for detecting target nucleic acids. The system includes a user interface for specifying a target and a data collection for storing a plurality of data including oligonucleotide sets corresponding to a plurality of known targets. The system further includes software for identifying any oligonucleotide sets in the data collection that are candidates for detecting the specified target and a plurality of quality metrics for scoring each identified oligonucleotide set. Each quality metric is assigned a default weight, and the weight of each quality metric is adjustable via the user interface.

According to another embodiment, a data collection includes nucleic acid sequences for a plurality of variants of a target. The data collection further includes a multiple alignment of the nucleic acid sequences for the plurality of variants of the target.

According to a still further embodiment, a database for storing data includes oligonucleotides corresponding to known targets, or complements thereof. The database further includes at least one score for indicating the suitability of each oligonucleotide for detecting at least one of the known targets.

According to a further embodiment, a computer-implemented system is provided for identifying oligonucleotide sets for detecting target nucleic acids. The system includes software for selecting oligonucleotides for detecting target nucleic acids and a database for storing data. The database includes data indicative of oligonucleotide sets corresponding to a plurality of known targets, or complements thereof, and for each target, data relating to decisions for selecting oligonucleotides for detecting the respective target. The software includes code for writing to the database data relating to decisions for selecting oligonucleotides for a particular target.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1 is a block diagram of a software system according to an illustrative embodiment of the invention;

FIG. 2 is a block diagram showing various ways in which the software system of FIG. 1 can be implemented on a computer network;

FIG. 3 is a flowchart showing how the software of FIG. 1 can be employed to generate ranked oligonucleotide sets for a particular amplification and/or detection technology;

FIG. 4 is a flowchart showing how the software of FIG. 1 can be employed to evaluate a user-specified oligonucleotide set;

FIG. 5 is a flowchart showing how the software of FIG. 1 can be employed to generate ranked combinations of oligonucleotide sets to detect a set of targets via a multiplex reaction; and

FIG. 6 provides a list of exemplary probe and primer consensus sequences comprising degenerate nucleotides, where x=A, G, C, T, or U, or functional equivalent.

### DETAILED DESCRIPTION OF THE INVENTION

Unless defined otherwise, all technical and scientific terms used herein have the same meaning as commonly understood by those of ordinary skill in the art to which this invention pertains. For convenience, the meaning of certain terms and phrases employed in the specification, examples, and appended claims are provided below to assist the reader in the practice of the invention.

The terms “homology” or “identity” or “similarity” refer to sequence relationships between two nucleic acid molecules and can be determined by comparing a nucleotide position in each sequence when aligned for purposes of comparison. The term “homology” refers to the evolutionary relatedness of two nucleic acid or protein sequences. The term “identity” refers to the degree to which nucleic acids are the same between two sequences. When a nucleotide position in the compared sequence is occupied by the same base, then the molecules are identical at that position. The term “similarity” refers to the degree to which nucleic acids are the same, but includes neutral degenerate nucleotides that can be substituted within a codon without changing the amino acid identity of the codon, as is well known in the art. An “unsimilar”, “unidentical” or “non-homologous” sequence shares less than about 40% identity, though preferably less than about 25% identity, with one of the target sequences of the present invention. Alternatively, percentage identity, homology or similarity are determined by the number of nucleotide differences in a sequence of a certain length. For example, a 100 nucleotide sequence with 20 nucleotide differences is defined as 80% identical, wherein a difference means a different nucleotide or absence of a nucleotide.

The phrase “substantial sequence identity” refers to two or more sequences or sub-sequences that have at least about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 100% nucleotide identity, as determined by visual inspection or alignment. Two nucleic acid sequences can be compared over their full-length (e.g., the length of the shorter of the two sequences, if they are of substantially different lengths) or over a portion of the sequences. Substantial sequence identity also exists when two nucleic acids hybridize to each other, typically requiring the annealing of at least about 6 contiguous nucleotides from each nucleic acid.

The term “Tm” means the temperature at which a population of double-stranded nucleic acid molecules becomes half-dissociated into single strands. Methods for calculating the Tm of nucleic acids are well known in the art (see, e.g., Berger and Kimmel (1987) Meth. Enzymol., Vol. 152: Guide To Molecular Cloning Techniques, San Diego: Academic Press, Inc. and Sambrook et al. (1989) Molecular Cloning: A Laboratory Manual, (2nd ed.) Vols. 1-3, Cold Spring Harbor Laboratory). As indicated by standard references, a simple estimate of the Tm value may be calculated by the equation: Tm=81.5+0.41 (% G+C), when a nucleic acid is in aqueous solution at 1 M NaCl (see, e.g., Anderson and Young, “Quantitative Filter Hybridization” in Nucleic Acid Hybridization (1985)). Other references include more sophisticated computations that take structural as well as sequence characteristics into account for the calculation of Tm. The Tm of a hybrid is affected by various factors such as the length and nature (e.g., DNA, RNA, base composition) of the nucleic acid and of the target, whether present in solution or immobilized), and the concentration of salts and other components (e.g., formamide, dextran sulfate, and polyethylene glycol). The effects of these factors are well known and are discussed in standard references in the art, see, e.g., Sambrook, supra, and Ausubel, supra.

Typically, hybridization conditions are salt concentrations less than about 1.0 M sodium ion, typically about 0.01 M to about 1.0 M sodium ion at about pH 7.0 to about 8.3, and temperatures at least about 30° C. for short probes (e.g., about 6 to about 50 nucleotides) and at least about 60° C. for long probes (e.g., greater than about 50 nucleotides). Appropriate stringency conditions that promote DNA hybridization, for example, about 2.0 to about 6.0× sodium chloride/sodium citrate (SSC) at about 45° C., followed by a wash of about 2.0×SSC at about 50° C., are known to those skilled in the art or can be found in Current Protocols in Molecular Biology, John Wiley & Sons, N.Y. (1989), sections 6.3.1-6.3.6. The salt concentration in the wash step can be selected from a low stringency of about 6.0×SSC to a high stringency of about 0.1×SSC. In addition, the temperature in the wash step can be performed at low stringency conditions at room temperature (i.e., about 22° C.), to high stringency conditions at about 65° C. Formamide can be added to the hybridization steps and washing steps in order to decrease the temperature requirement by 1° C. per 1% formamide added. The phrase “stringent hybridization conditions” generally refers to conditions in a range from about 5° C. to about 20° C. or 25° C. below the melting temperature (Tm) of the target sequence.

The phrase “substantially pure” or “isolated,” when referring to nucleic acids, generally refers to the nucleic acid separated from contaminants with which it is generally associated, e.g., lipids, proteins and other nucleic acids. The substantially pure or isolated nucleic acids of the present invention will be greater than about 50% pure. Typically, these nucleic acids will be more than about 60% pure, more typically, from about 75% to about 90% pure and preferably from about 95% to about 98% pure.

Methods for Designing Primers or Probes

The methods of the invention may be performed manually but may also be performed by a software program referred to herein as PriMD™ software. Details of how the methods may be performed are described below.

### Identifying a Conserved Region(s)

A gene or genomic region that is the best conserved or representative of a particular target, such as an organism, infectious agent, mutation, or polymorphism is chosen. This conserved region need only have two or three runs of 15-40 sequential nucleotides within a 50 to 300 nucleotide region, for example. Genes or genomes that have been sequenced more frequently may provide a better indication of genetic variability. If there is not enough information in the scientific literature, an alignment can be performed for each gene in a given target. A plot of conservation against nucleotide position provides a good indication of candidate regions. In an embodiment, this step is performed manually using either dedicated databases (e.g., Influenza Sequence Database or the Ribosomal Database Project). In another embodiment, the step is performed by taking a Genbank reference sequence and performing a BLAST analysis, or the equivalent, to identify all related sequences. In another embodiment, all publicly available sequences associated with a target are located in, or entered into, a database and are each annotated with as much pertinent information as is available to provide parameters for selecting the optimal sequences. Such a database also contains all the possible sequences that might be present along with the target. For example, if the target is Influenza A virus, the database screens any candidate Influenza A primers or probes against other organisms known to be present in the respiratory tract (such as other viruses, bacteria, normal host flora and fauna) as well as relevant host genetic markers so that cross hybridizing sequences can be excluded.

Alignments

In an embodiment, one sequence acts as a reference sequence, to which test (e.g., other variant) sequences are compared and aligned. When using a sequence comparison algorithm, test and reference sequences are input into a computer, sub-sequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Natl. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols In Molecular Biology, Greene Publishing and Wiley-Interscience, New York (supplemented through 1999). Each of these references and algorithms is incorporated by reference herein in its entirety. When using any of the aforementioned algorithms, the default parameters for window length, gap penalty, etc., are generally used.

In an embodiment, sequences that relate to the conserved gene or region are imported into a storage file such as, for example, a FastA file, and imported into an alignment program, such as, for example, ClustalW, to perform a multiple sequence alignment. The file may be edited to remove extraneous nucleotides at the ends as well as sequences that clearly do not align, for example, using the GenDoc program. If sequences are removed, the multiple sequence alignment is repeated. For targets that have a limited number of sequences there are alternative programs that provide more exhaustive alignments (e.g., a pair-wide analysis using evolution scoring, entropy scoring, consistency scoring or “traveling salesman” scoring). However, once the number of sequences gets large (e.g., over 100) or the sequences themselves are large (e.g., over 5000 bases), there are very few alternatives to the ClustalW program.

Consensus Sequence

A consensus sequence is then chosen as the target sequence for selecting primers and/or probes. Both strands are typically analyzed and any duplicates are eliminated. A PCR penalty formula may be used to identify a pair of optimal primers and, e.g., an internal probe for TaqMan® Real Time PCR, such as a weighted sum of the following measurements: (1) Tm—Optimal Tm of the primers; (2) Difference Between Primer Tms; (3) Amplicon Length; and (4) Distance Between Primer And Taqman® Probe.

The target sequence is checked for every available primer or probe binding site and assigns the candidate primers and probes are assigned a score based on the certain parameters, for example: primer melting temperature (Tm)—optimum about 59° C., with a range of about 58° C. to about 60° C., but each pair must not differ by more than about 1° C.; primer composition—about 30% to about 80% GC; primer length—about 9 bases to about 40 bases; primer secondary structure; and amplicon length (any length up to 250 bases); and Tm—about 0° C. to about 85° C.; primers with runs of four or more identical nucleotides, especially G, are rejected; and the total number of Gs and Cs in the last five nucleotides at the 3′ end of a primer should not exceed two. Probes will have a melting temperature about 10° C. higher than the primers. Probes with a G at the 5′ end are rejected as the G can quench reporter fluorescence even after cleavage. There should also be more Cs than Gs in the probe. These parameters are designed such that any resulting set of primers and probe will be capable of efficient PCR. The parameters are relaxed (e.g., amplicon size is increased, primer Tm differences are increased, etc.) if a good set of primers and probe is not identified based on their ability to identity rank.

“Exclude/Include” Function

All the sequences in the database can be assigned to the Exclude/Include function of Primer3. For example, the sequences that are used to generate the consequence sequence for a target form part of the Include file. Once the consensus sequence for a target is selected, sequences in the database that were not used for generating the consensus can become part of the Exclude file. The sequences in the database not only represent potential targets but also sequences from organisms that could be expected to be present in an experimental sample as well as all closely-related organisms that might cause false positive results. If a target requires multiple sets of primer & probe, as each set is identified, they would become part of the Exclude file for subsequent primer & probe sets (see section entitled Multiplexing). In other words, every primer or probe chosen by the methods and software of the invention will have been BLASTed or screened against the Exclude file to eliminate mis-priming or false-positive results. There are different stages in the selection process when this functionality can be performed. For example, rather than screen every possible primer and probe, the Exclude function may be run against the best 1000 sets, for example, of primers and probe.

Score Assignment

Each of the sets of primers and probes selected will be ranked by a combination of methods as individual primers and probes and as a primer/probe set. This will involve one or more method of ranking (e.g., joint ranking, hierarchical ranking, and serial ranking) where sets of primers and probes will be eliminated or included based on any combination of the following criteria, and a weighted ranking again based on any combination of the following criteria, for example: (A) Percentage Identity to Target Variants; (B) Conservation Score; (C) Coverage Score; (D) Strain/Subtype/Serotype Score; (E) Associated Disease Score; (F) Duplicates Sequences Score; (G) Year and Country of Origin Score; (H) Patent Score, and (I) Epidemiology Score.

A. Percentage Identity

A percentage identity score is based upon the number of target nucleic acid variant (e.g., native) sequences that can hybridize with perfect conservation (the sequences are perfectly complimentary) to each primer or probe of a primer pair & probe set. If the score is less than 100%, the program ranks additional primer pair & probe sets that are not perfectly conserved. This is a hierarchical scale for percent identity starting with perfect complimentarity, then one base degeneracy through to the number of degenerate bases that would provide the score closest to 100%. The position of these degenerate bases would then be ranked. The methods for calculating the conservation is described under section B.

(i) Individual Base Conservation Score

A set of conservation scores is generated for each nucleotide base in the consensus sequence and these scores represent how many of the target nucleic acid variants sequences have a particular base at this position. For example, a score of 0.95 for a nucleotide with an adenosine, and 0.05 for a nucleotide with a cytidine means that 95% of the native sequences have an A at that position and 5% have a C at that position. A perfectly conserved base position is one where all the target nucleic acid variant sequences have the same base (either an A, C, G, or T/U) at that position. If there is an equal number of bases (e.g., 50% A & 50% T) at a position, it is identified with an N.

(ii) Candidate Primer/Probe Sequence Conservation

An overall conservation score is generated for each candidate primer or probe sequence which represents how many of the target nucleic acid variant sequences will hybridize to the primers or probes. The program assumes that perfectly complimentary sequences are superior to mismatched sequences when hybridizing to a complimentary target nucleic acid variant sequence. A candidate sequence that is perfectly complimentary to all the target nucleic acid variant sequences will have a score of 1.0 and rank the highest.

For example, illustrated below are three different 10-base candidate probe sequences that are targeted to different regions of a consensus target nucleic acid variant sequence. Each candidate probe sequence is compared to a total of 10 native sequences.

#1. A A A C A C G T G C
0.7 1.0 1.0 1.0 1.0 1.0 1.0 1.0 1.0 1.0

Number of target nucleic acid variant sequences that are perfectly complimentary—7. Three out of the ten sequences do not have an A at position 1.

#2. C C T T G T T C C A
1.0 0.9 1.0 0.9 0.9 1.0 1.0 1.0 1.0 1.0

Number of target nucleic acid variant sequences that are perfectly complimentary—7, 8, or 9. At least one target nucleic acid variant does not have a C at position 2, T at position 4, or G at position 5. These differences may all be on one target nucleic acid variant molecule or may be on two or three separate molecules.

#3. C A G G G A C G A T
1.0 1.0 1.0 1.0 1.0 0.9 0.8 1.0 1.0 1.0

Number of target nucleic acid variant sequences that are perfectly complimentary—7 or 8. At least one target nucleic acid variant does not have an A at position 6 and at least two target nucleic acid variant do not have a C at position 7. These differences may all be on one target nucleic acid variant molecule or may be on two separate molecules.

A simple arithmetic mean for each candidate sequence would generate the same value of 0.985. However, the number of target nucleic acid variant sequences identified by each candidate probe sequence can be very different. Sequence #1 can only identify 7 native sequences because of the 0.7 (out of 1.0) score by the first base—A. Sequence #2 has three bases each with a score of 0.9; each of these could represent a different or shared target nucleic acid variant sequence. Consequently, Sequence #2 can identify 7, 8 or 9 target nucleic acid variant sequences. Similarly, Sequence #3 can identify 7 or 8 of the target nucleic acid variant sequences. Therefore, Sequence #2 would be the best choice if all the three bases with a score of 0.9 represented the same 9 target nucleic acid variant sequences.

(iii) Overall Conservation Score of the Primer & Probe Set—Percent Identity

The same method described in (ii) when applied to the complete primer pair & probe set will generate the percent identity for the set (see A above). For example, using the same sequences illustrated above, if Sequences #1 & #2 are primers and Sequence #3 is a probe, then the percent identity for the target can be calculated from how many of the target nucleic acid variant sequences are identified with perfect complimentarity by all three primer/probe sequences. The percent identity could be no better than 0.7 (7 out of 10 target nucleic acid variant sequences) but as little as 0.1 if each of the degenerate bases reflects a different target nucleic acid variant sequence. Again, an arithmetic mean of these three sequences would be 0.985. As none of the above examples were able to capture all the target nucleic acid variant sequences because of the degeneracy (scores of less than 1.0), the ranking system takes into account that a certain amount of degeneracy can be tolerated under normal hybridization conditions, for example, during a polymerase chain reaction. The ranking of these degeneracies is described in (iv) below.

An in silico evaluation determines how many native sequences (e.g., original sequences submitted to public databases) are identified by a given candidate primer/probe set. The ideal candidate primer/probe set is one that can perform PCR and the sequences are perfectly complimentary to all the known native sequences that were used to generate the consensus sequence. If there is no such candidate, then the sets are ranked according to how many degenerate bases can be accepted and still hybridize to just the target sequence during the PCR and yet identify all the native sequences.

In another example, addition probes can be designed by PriMD that will hybridize to all the native sequences that are not recognized by the first probe. The same primer pair can be used for all probes. The multiple probes will be designed to function as a multiplex reaction.

In another example, addition sets of primers & probes can be designed by PriMD that will hybridize to all the native sequences that are not recognized by the first set of primers & probe. The sets will be designed to function as a multiplex reaction.

The hybridization conditions, for TaqMan as an example are: 10-50 mM Tris-HCl pH 8.3, 50 mM KCl, 0.1-0.2% Triton® X-100 or 0.1% Tween®, 1-5 mM MgCl₂. The hybridization is performed at 58-60° C. for the primers and 68-70° C. for the probe. The in silico PCR identifies native sequences that are not amplifiable using the candidate primers & probe set. The rules can be as simple as counting the number of degenerate bases to more sophisticated approaches based on exploiting the PCR criteria used by the PriMD™ software. Each target nucleic acid variant sequence has a value or weight (see Score assignment above). If the failed target nucleic acid variant sequence is medically valuable, the primer/probe set is rejected. This in silico analysis provides a degree of confidence for a given genotype and is important when new sequences are added to the databases. New target nucleic acid variant sequences are automatically entered into both the “include” and “exclude” categories. For example, a new Influenza A sequence is tested against an Influenza Virus A primer/probe set of the invention in the include category but will be added to the exclude category when it is tested against other primer/probe sets, such as Influenza Virus. Published primer & probes will also be ranked by the PriMD software.

(iv) Position (5′ to 3′) of the Base Conservation Score

In an embodiment, primers should not have any bases in the terminal five positions at the 3′ end with a score less than 1. This is one of the last parameters to be relaxed if the method fails to select any candidate sequences. The next best candidate having a perfectly conserved primer would be one where the poorer conserved positions are limited to the terminal bases at the 5′ end. The closer the poorer conserved position is to the 5′ end, the better the score. For probes, the position criteria is different. For example, with a TaqMan® probe, the most destabilizing effect occurs in the center of the probe. The 5′ end of the probe is also important as this contains the reporter molecule that must be cleaved, following hybridization to the target, by the polymerase to generate a sequence-specific signal. The 3′ end is less critical. Therefore, a sequence with a perfectly conserved middle region will have the higher score. The remaining ends of the probe are ranked in a similar fashion to the 5′ end of the primer. Thus, the next best candidate to a perfectly conserved TaqMan® probe would be one where the poorer conserved positions are limited to the terminal bases at either the 5′ or 3′ ends. The hierarchical scoring will select primers with only one degeneracy first, then primers with two degeneracies next and so on. The relative position of each degeneracy will then be ranked favoring those that are closest to the 5′ end of the primers and those closest to the 3′ end of the TaqMan probe. If there are two or more degenerate bases in a primer and probe set the ranking will initially select the sets where the degeneracies occur on different sequences.

B. Coverage Score

The total number of aligned sequences is considered under coverage score. A value is assigned to each position based on how many times that position has been reported or sequenced. Alternatively, coverage can be defined as how representative the sequences are of the known strains, subtypes etc., or their relevance to a certain diseases. For example, the target nucleic acid variant sequences for a particular gene may be very well conserved and show complete coverage but certain strains are not represented in those sequences.

A sequence is included if it aligns with any part of the consensus sequence (which is usually a whole gene or a functional unit) or has been described as being a representative of this gene. Even though a base position is perfectly conserved it may only represent a fraction of the total number of sequences (for example, if there are very few sequences). For example, region A of a gene shows a 100% conservation from 20 sequence entries while region B in the same gene shows a 98% conservation but from 200 sequence entries. There is a relationship between conservation and coverage if the sequence shows some persistent variability. As more sequences are aligned, the conservation score falls, but this effect is lessened as the number of sequences gets larger. Unless the number of sequences is very small (e.g., under 10) the value of the coverage score is small compared to that of the conservation score. To obtain the best consensus sequence, artificial spaces are allowed to be introduced. Such spaces are not considered in the coverage score.

D. Strain/Subtype/Serotype Score

A value is assigned to each strain or subtype or serotype based upon its relevance to a disease. For example, strains of INF-A that are linked to pandemics will have a higher score than strains that are generally regarded as benign or included in the current vaccine. The score is is based upon sufficient evidence to automatically associate a particular strain with a disease. For example, certain strains of adenovirus are not associated with diseases of the upper respiratory system. Accordingly, there will be sequences included in the consensus sequence that are not associated with diseases of the upper respiratory system.

E. Associated Disease Score

The associated disease score pertains to strains that are not known to be associated with a particular disease (to differentiate from D above). Here, a value is assigned only if the submitted sequence is directly linked to the disease and that disease is pertinent to the assay.

F. Duplicate Sequences Score

If a particular sequence has been sequenced more than once it will have an effect on representation, for example, a strain that is represented by 12 entries in Genbank of which six are identical and the other six are unique. Unless the identical sequences can be assigned to different strains/subtypes (usually by sequencing other gene or by immunology methods) they will be excluded from the scoring.

G. Year and Country of Origin Score

The year and country of origin scores are important in terms of the age of the human population and the need to provide a product for a global market. For example, strains identified or collected many years ago may not be relevant today. Furthermore, it is probably difficult to obtain samples that contain these older strains. In addition, some strains may have the potential for creating an epidemic if most of the present population does not have immunity (e.g., certain influenza A strains). Certain divergent strains from more obscure countries or sources may also be less relevant to the locations that will likely perform clinical tests, or may be more important for certain countries (e.g., North America, Europe, or Asia).

H. Patent Score

Candidate target variant sequences published in patents are searched electronically and annotated such that patented regions are excluded. Alternatively, candidate sequences are checked against a patented sequence database.

I. Minimum Qualifying Score

The minimum qualifying score is determined by expanding the number of allowed mismatches in each set of candidate primers and probes until all possible native sequences are represented (i.e., has a qualifying hit).

J. Other

A score is given to based on other parameters, such as relevance to certain patients (e.g., pediatrics, immunocompromised) or certain therapies (e.g., target those strains that respond to treatment) or epidemiology. The prevalence of an organism/strain and the number of times it has been tested for in the community can add value to the selection of the candidate sequences. If a particular strain is more commonly tested then selection of it would be more likely. Strain identification can be used to selection better vaccines.

Primer/Probe Evaluation

Once the candidate primers and probes have received their scores and have been ranked, they are evaluated using any of a number of methods of the invention, such as BLAST analysis and secondary structure analysis.

A. BLAST Analysis

The candidate primer/probe sets are submitted to BLAST analysis to check for possible overlap with any published sequences that might be missed by the Include/Exclude function. It also provides a useful summary.

B. Secondary Structure

The methods and software of the invention can also incorporate an analysis of nucleic acid secondary structure. This includes the structures of the primers and/or probes as well as their intended target variant sequences. The methods and software of the invention predict the optimal temperatures for the annealing but assumes that the target (e.g., RNA or DNA) does not have any significant secondary structure. For example, if the starting material is RNA, the first stage is the creation of a complimentary strand of DNA (cDNA) using a specific primer. This is usually performed at temperatures where the RNA template can have significant secondary structure thereby preventing the annealing of the primer. Similarly, after denaturation of a double stranded DNA target (for example, an amplicon after PCR), the binding of the probe is dependent on there being no major secondary structure in amplicon.

The methods and software of the invention can either use this information as a criteria for selecting primers and probes or evaluate any secondary structure of a selected sequence, for example, by cutting and pasting candidate primer or probe sequences into a commercial internet link that uses software dedicated to analyzing secondary structure, such as, for example, MFOLD (Zuker et al. (1999) Algorithms and Thermodynamics for RNA Secondary Structure Prediction: A Practical Guide in RNA Biochemistry and Biotechnology, J. Barciszewski and B. F. C. Clark, eds., NATO ASI Series, Kluwer Academic Publishers).

C. Evaluating the Primer and Probe Sequences

The methods and software of the invention may also analyze any nucleic acid sequence to determine its suitability in a nucleic acid amplification-based assay. For example, it can accept a competitor's primer set and determine the following information: (1) How it compares to the primers of the invention (e.g., overall rank, PCR & conservation ranking, etc.); (2) How it aligns to the Exclude Libraries (e.g., assessing cross-hybridization)—also used to compare primer and probe sets to newly published sequences; and (3) If the sequence has been previously published. This step requires keeping a database of sequences published in scientific journals, posters, and other presentations.

Multiplexing

The Exclude/Include capability is ideally suited for designing multiplex reactions. The parameters for designing multiple primer and probe sets adhere to a more stringent set of parameters than those used for the initial Exclude/Include function. Each set of primers & probe, together with the resulting amplicon is screened against the other sets that constitute the multiplex reaction. As new targets are accepted their sequences are automatically added to the Exclude category.

The database is designed to interrogate the online databases to determine and acquire, if necessary, any new sequences relevant to the targets. These sequences are evaluated against the optimal primer/probe set. If they represented a new genotype or strain then a multiple sequence alignment may be required.

Software System of the Invention

As used herein and particularly in the claims, the term “software” is defined broadly as any computer-readable code, whether compiled or uncompiled, that performs a function in a computer or other computational system. “Software” can thus include a single line of code or a single encoded expression. It can also include larger modules or sections, code distributed among different modules or sections, and larger software systems and applications.

The software of the invention, referred to herein as the PriMD™ software, enables a user to automate the selection of primer and probe sets described above. For example, the PriMD™ software can design primers, probes, primer sets, and primer/probe sets to identify groups of genes that represent strains of infectious organisms or other disease related genes. The PriMD™ software is an efficient, high-throughput, automatic system that produces and evaluates millions of primer and/or probe set combinations. Given an alignment of target variant sequences and a set of sequences to exclude, the PriMD™ software produces a ranked list of primer and/or probe sets that identify the target variants. Primer and/or probe sets are ranked by a combination of criteria, as described above, including percentage identity, PCR penalty, conservation, and coverage scores. In addition to designing primers, the PriMD™ software is linked to a database that stores key data of each instance of the running the software. The PriMD™ database allows the user to store the data and decisions that went into creating each primer and/or probe set. The PriMD™ database may be queried to ask useful questions, for example, to determine how current each primer and/or probe set is relative to new sequences appearing in the public sequence databases.

The PriMD™ Database

The database of the invention comprises all sequences relevant to the target variants sequences. This includes the derived consensus sequences for each target, all the sequences described for each target, all the host sequences, as well as any sequences that might be expected to be associated with the target. Each sequence has information regarding phylogeny (e.g., strain, subtype, and genotype), country of origin, source (i.e., type of infectious material), disease association, year, any patents linked to these sequences, plus notations if missing information or a duplicate sequences.

Software Components

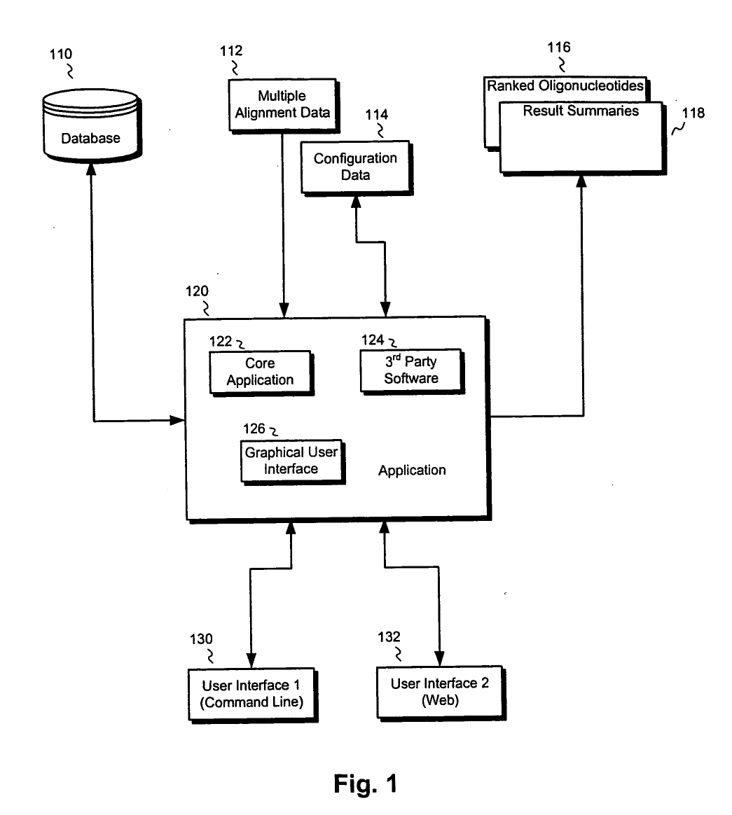

FIG. 1 shows an overview of a software system according to an illustrative embodiment of the invention. As shown in FIG. 1, the software system includes a data collection, such as database 110 (the PriMD™ Database). The database 110 is provided in communication with a software application 120, which has the ability both to read from and write to the database 110. The software application 120 is further provided in communication with input data sources 112 and 114, for receiving data, and with output data locations 116 and 118.

In one embodiment, the software application 120 is installed on a computer running the Linux operating system. The software system 120 is made available to users via two user interfaces: a first user interface 130 and a second user interface 132. The first user interface 130 is a Linux command line interface. This interface receives commands entered manually by users and outputs data to the users' computer screens. Users of this interface are generally local to the computer; however they may also access the computer remotely, such as via a remote control program or terminal emulation program. The second interface 132 is a web interface. This interface provides access to users via HTTP. The web interface includes the user's web browser and may be accessed over the Internet.

The database 110 is preferably a relational database, such as an Oracle, MySQL, or SQL Server database. However, this is not required. Alternatively, any form of data collection can be used, such as a spreadsheet, a collection of spreadsheets, an XML file, a collection of XML files, and so forth. In one embodiment, the database 110 is implemented as a collection of text files saved in a directory structure.

The input data source 112 is preferably a multiple alignment file. A suitable example of this type of file is a FastA file generated by a Clustal computer program. Other file formats and/or computer programs may be used. In addition, multiple alignment data need not be provided in the form of a file. For example, the data can also be stored in one or more fields of a database (including the database 110) or manually entered by a user.

The input data source 114 is a configuration file. This file preferably contains a list of all quality metrics associated with scoring and/or ranking different oligonucleotides and oligonucleotide sets, ideal values for each quality metric, and weighting factors to be applied to each quality metric. Preferably, the file provides default values for the weighting factors. Users can vary these values from their defaults via controls on the first and/or second user interface. In one embodiment, the data source 114 is provided as part of the database 110, and no separate file is required.

Output data 116 and 118 are preferably stored in files. Output data 116 lists ranked oligonucleotide sets for users to examine. Output data 118 provides results of a run of the software in summary form. These data may be accessed, via the user interface 130 or 132, and displayed on a user's computer screen. Local users can also access these files directly via the Linux file system.

The software application 120 preferably includes various components. These can be broadly classified in three categories: a core application 122, third party software (including modifications thereof) 124, and GUI (graphical user interface) software 126 for managing HTTP communications.

The core application 122 performs numerous functions associated with the design and evaluation of oligonucleotides. In one embodiment, the core application 122 is a collection of classes written in object-oriented Perl. This collection may include the following components:
A main driver class that invokes other classes
A class that generates valid singleplex oligonucleotide sets
A class that generates multiplex combinations of oligonucleotide sets
A class for evaluating third party oligonucleotide sets
A class for communicating with the database 110
A class for excluding oligonucleotides and/or oligonucleotide sets
A class for evaluating in silico PCR
A class for communicating with a modified version of Primer3
A class for ranking oligonucleotide sets and multiplex combinations of oligonucleotide sets in multiple ways
A class for each amplification/detection technology (e.g., TaqMan PCR)

In addition, the third party software 124 may include the following components:
A modified version of Primer3
BioPerl
Clustal/GeneDoc
Blast
Software for secondary structure
Apache Web Server

Moreover, the GUI software 126 may include the following components:
A main CGI program
A main Java-based servlet
Code for presenting information from the database 110
Code for accepting user input
Code for graphing
Code for report generation
Perl presentation classes
Java presentation classes

The components of the software system of FIG. 1 may all reside on a single computer. However, the software system is not limited to this arrangement.

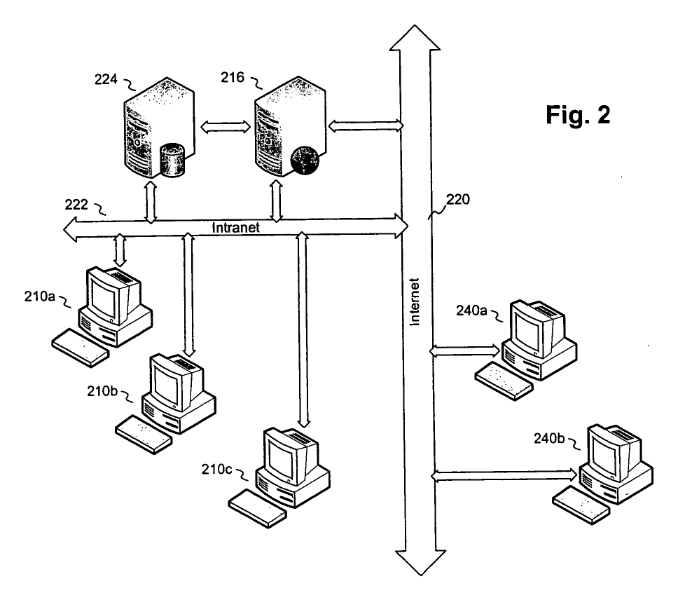

FIG. 2 shows a variety of other arrangements for implementing the software system of FIG. 1. In one arrangement, the database 110 is installed on a database server 224, and the software application 120 is installed on a web server 216. The software application 120 communicates with the database 110 via an intranet 222. Computers, such as computers 210a-210c, access the software application 120 via the intranet 222 using web browsers. Computers outside the intranet also access the system. For instance, computers 240a and 240b can access the web server 216 via the Internet 222.

In another arrangement, the database server 224 and web server 216 are combined into a single server. The entire application, including the database, can thus be served from a single computer.

The components of the software system may be distributed and accessed in numerous ways. Those shown in FIG. 2 are provided merely for illustration and are not intended to limit the scope of the invention.

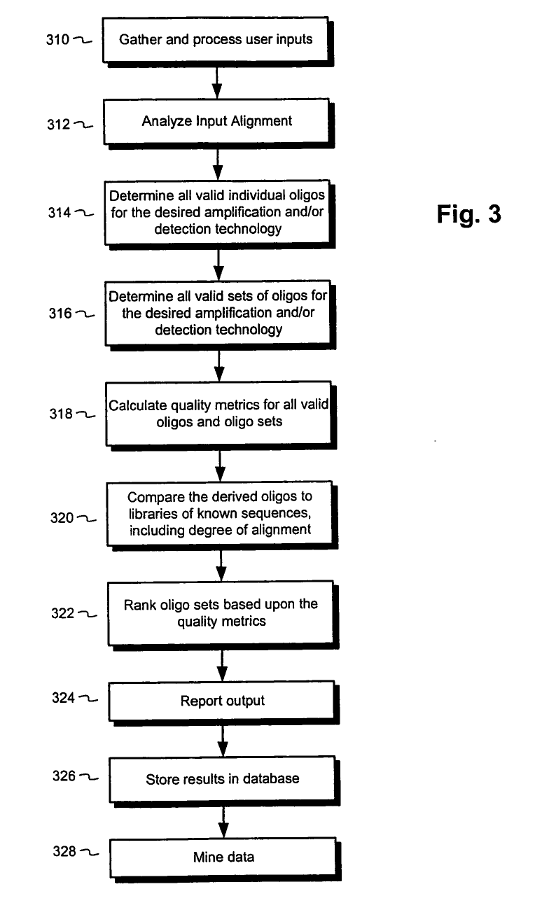

FIGS. 3-5 show various processes that the software system of FIG. 1 can preferably conduct. These processes are provided as examples and are not intended as an exhaustive list of the software system's capabilities.

FIG. 3 shows a process for generating ranked oligonucleotide sets for a particular amplification and/or detection technology. At step 310, the software gathers and processes user inputs. The inputs include the multiple alignment data 110, which provide a multiple alignment of different variants of a target nucleic acid sequence for which primers and/or probes are to be identified. The inputs may optionally include other data, such as exclude data, e.g., sequences to which oligonucleotides should not align, as well as market data, patient demographics, information about each target sequence (such as strain), geographical considerations, and importance.

At step 312, the software analyzes the multiple alignment data. This step includes generating a representative sequence from the multiple alignment data. The “representative sequence” is similar to the consensus sequence, described above. It differs from the consensus sequence in that the representative sequence contains no unknowns (X's). Each base position is assigned a value, one of A, T, C, or G. The value assigned to any base position is the value that occurs most frequently for that base position in the multiple alignment data.

At step 314, the software determines all valid individual oligonucleotides for the desired amplification and/or detection technology. This step preferably includes computing each possible oligonucleotide (e.g., each forward primer, each reverse primer, and each probe) that could validly hybridize with the representative sequence given the requirements of the amplification and/or detection technology. All strands that are complementary to the representative sequence and that meet the chemical and informatic requirements for oligonucleotides of the selected process are preferably identified. In addition, the software preferably filters out any sequences identified in the exclude file at this time.

At step 316, the software constructs sets of oligonucleotides identified in step 314. Each set is assembled such that it works together as a whole in a manner consistent with the requirements of the desired amplification and/or detection technology. For example, a set assembled for TaqMan must include one oligonucleotide that is suitable as a TaqMan forward primer, one oligonucleotide that is suitable as a TaqMan reverse primer, and one oligonucleotide that is suitable as a TaqMan probe. The software preferably considers additional chemical and informatic factors for the sets, such as whether any oligonucleotides in a set cross-hybridize with any other oligonucleotides in the set.

At step 318, the software calculates at least one quality metric for all valid oligonucleotides sets. Preferably, the software scores each oligonucleotide set and each individual oligonucleotide included in each set produced by step 316 for each of the quality metrics defined by the configuration data 114, which are identified as “criteria” under “Score Assignment” above.

At step 320, the software compares oligonucleotide identified at step 314 with libraries of known sequences. An objective of this step is to determine whether any identified oligonucleotides are likely to hybridize with targets other than the desired target and its variants. This step thus gives important information about whether any of the identified oligonucleotides might cause a false positive result when included in a diagnostic kit. The software preferably assigns each oligonucleotide a score based on its likelihood of generating a false positive result.

Another objective of this step is to ascertain whether any of the identified oligonucleotides are patented. Patents on oligonucleotides can present obstacles to use. The software preferably assigns each oligonucleotide a patent score depending onto whether it is protected by one or more patents. To complete this step, the software preferably runs a program, such as BLAST, for automatically determining a degree of homology between each identified oligonucleotide and all sequences stored in each respective library and for obtaining patent information. Various libraries can be used, including GenBank, Derwent, and the database 110 (the PriMD™ Database).

At step 322, the software ranks the oligonucleotide sets determined at step 316 based upon the scores they received for the quality metrics. Various types of rankings can be performed, such as joint ranking, hierarchical ranking, serial ranking, and ranking that measures the dissimilarity between actual metric scores and ideal scores. These are described in more detail below. The software is preferably user-configurable to rank the oligonucleotide sets based on a subset of quality metrics (including a single metric), or based on all of the quality metrics.

The purpose of ranking is to present to the user a collection of oligonucleotide sets that are most suitable for a diagnostic assay, in the sense that the oligonucleotide sets best detect most or all of the variants of the target. Ranking is based upon a set of desirable oligonucleotide set characteristics or criteria. These characteristics may sometimes be in competition with one another, in that maximizing one characteristic may not maximize the other. The goal of ranking is to identify the degree to which each oligonucleotide set maximizes all the desired characteristics or best balances the tradeoffs between these characteristics, and to then sort the sets accordingly. Another goal of ranking is to determine all pertinent data about the suitability of each oligonucleotide set, thereby allowing the user to understand the tradeoffs between possibly competing characteristics. Based upon the various ranking produced by the software system, the user may select the single best oligonucleotide set (or collection of sets) that represents an optimal balance of desired characteristics in accordance to the user's preferences. Towards that end, the user can specify alternative degrees of importance of various characteristics (e.g., in the form of weights) that override default settings.

At step 324, the software reports the results of the run to the user. These results include the ranked oligonucleotides 116 and the results summaries 118 described in connection with FIG. 1.

At step 326, the software stores various information derived from its run in the database 110. Examples of this stored information include:
The multiple alignment data gathered at step 310
The consensus sequence
The representative sequence
List of best ranked oligonucleotide sets
Weights used for each quality metric
Scores for each oligonucleotide and oligonucleotide set for each of the quality metrics, including conservation, coverage, and other alignment-related criteria
Any excluded oligonucleotides
Date on which the software was run.

An objective of saving this data in the database 110 is to provide a record of the circumstances surrounding each run of the software. This record may be consulted as time passes to examine the rationale behind choosing certain oligonucleotide sets. It may also help to determine whether the circumstances surrounding the original software run have changed to an extent that the user may wish to rerun the software to generate a more current assortment of oligonucleotide sets.

At step 328, the user has the option of mining the data produced by the software system, e.g., interactively exploring the results to determine the most suitable oligonucleotide sets.

The process steps 310-328 need not follow the precise order depicted in FIG. 3. For example, the step 320 of comparing the derived oligonucleotides to libraries of known sequences may be conducted at any point after the step 314 of determining all valid individual oligonucleotides and before the step 322 of ranking the oligonucleotide steps. Similarly, the act of filtering all oligonucleotides set forth in the exclude file need not be conducted at step 314, as described above, but may be conducted at any point prior to step 322. The step 318 of calculating quality metrics need not be conducted all at once in a single step, but rather may be calculated as information becomes available. Thus, quality metrics related to alignment, such as conservation and coverage, can be computed as early as step 312 (Analyze input alignment). Similarly, metrics related to individual oligonucleotides can be computed at any point after step 314. Along a similar vein, there is no need to report output (step 324) before results are stored in the database (step 326). Results may just as well be reported after they are stored. Therefore, it should be understood that the order of steps set forth in FIG. 3 is not limiting but is merely an example how a process may be conducted according to the invention.

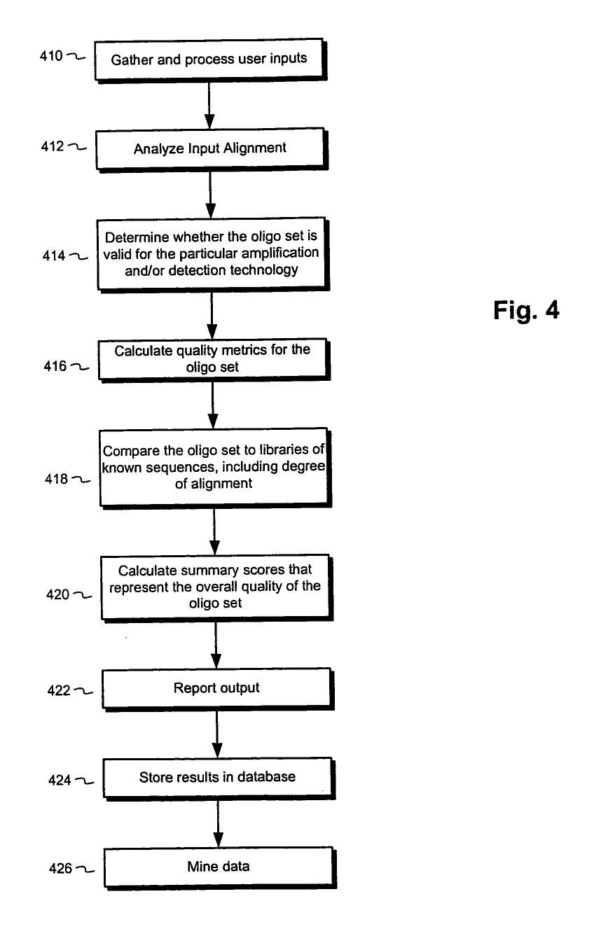

FIG. 4 shows a process for evaluating a user-specified oligonucleotide set, to determine its suitability for detecting a target sequence and its variants via a particular amplification and/or detection technology. This process is preferably similar to the process described in connection with FIG. 3, except that, in this case, a user supplies a particular oligonucleotide set and directs the software to score that set.

The process begins with the software gathering and processing user inputs (step 410) and analyzing input alignment (step 412). These steps are preferably similar to steps 310 and 312 described above.

At step 414, the software determines whether the user-specified oligonucleotide set is valid for the desired amplification and/or detection technology. This step includes determining whether the individual oligonucleotides meet the requirements of the desired process. Substantially the same methods are used in step 414 for determining validity of individual oligonucleotides as were set forth in connection with step 314 above. This step also includes determining whether the oligonucleotide set as whole meets the requirements of the desired process. Substantially the same methods are used for determining the validity of the oligonucleotide set as were set forth in connection with step 316 above.

At step 416, the software calculates quality metrics for the specified oligonucleotide set. This step is preferably similar to step 318 above, except that quality metrics need only be calculated for the one user-specified oligonucleotide set rather than for all valid sets.

At step 418, the software compares the specified oligonucleotide set to libraries of known sequences. This step is preferably similar to step 320 above, except that the software need only compare the user-specified oligonucleotide set to the libraries, rather than all derived oligonucleotide sets.

At step 420, the software calculates summary scores that represent the overall quality of the user-selected oligonucleotide set. The summary scores represent different ways of combining the scores on the individual quality metrics, e.g., different weighting or different algorithms or formulas used to generate the score, as described above. Steps 422, 424, and 426 of FIG. 4 are preferably similar to steps 324, 326, and 328 of FIG. 3.

As with FIG. 3, the order of steps depicted in FIG. 4 are provided for illustration and are not intended to limit the invention. The order of steps in FIG. 4 can be varied in ways similar to those discussed in connection with FIG. 3.

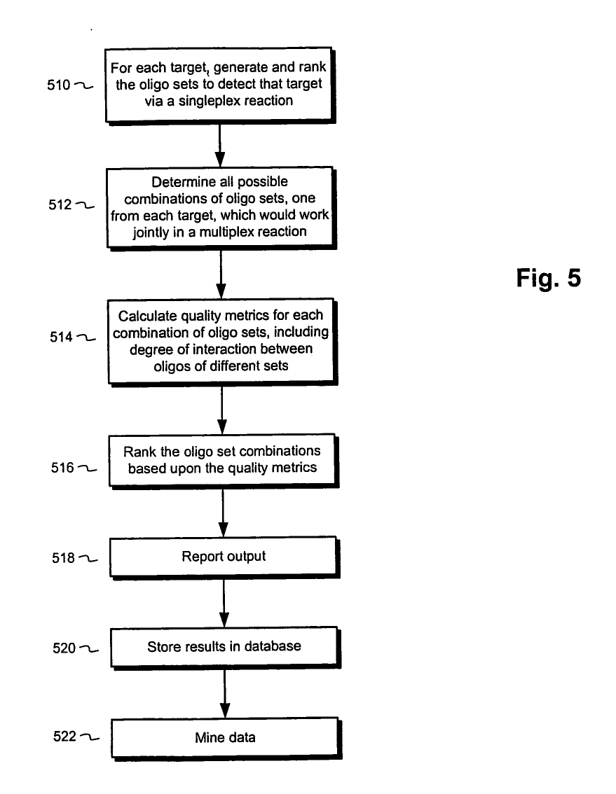

FIG. 5 shows a process for generating and ranking a combination of oligonucleotide sets to detect a set of different targets and their variants via a multiplex reaction.

At step 510, the software generates and ranks oligonucleotide sets for each target (and its variants) individually, as if for a singleplex reaction, using the process shown in FIG. 1. The process shown of FIG. 1 is thus repeated for each target that the user wishes to include in the multiplex reaction. At the completion of step 510, a different group of ranked oligonucleotide sets is produced for each target (and its variants).

At step 512, the software determines all possible combinations of oligonucleotide sets from the groups provided from step 510. To ensure that all targets are represented, each combination includes one oligonucleotide set from the group provided for each target.

At step 514, the software computes quality metrics for each combination of oligonucleotide sets produced from step 512. This step is similar to step 318 above, except that step 514 also computes one or more quality metrics relating to the degree of interaction between oligonucleotides for the different targets. These preferably include the likelihood of cross-hybridization, as well as other chemical and informatic factors relating to how well each combination works as a whole with the desired amplification and/or detection technology.

At step 516, the software ranks the combinations of oligonucleotide sets based upon the quality metrics. This step is similar to the ranking step 322 described in connection with FIG. 3 above

Steps 518-522, which relate to reporting output, storing results in the database, and mining data, are preferably similar to steps 324-328 described above.

Additional Software Matters

The workflow application invokes a series of steps in succession, reading from, or writing to, the database at key points. For example, when generating TaqMan® primers and probes, the software initially finds every possible primer and every possible probe. It then “puts them together” to create the best primer pair/probe set. However, each primer and probe that make up this best set may not necessarily be the best individual forward, reverse or probe sequence, i.e., the primer and probe set may not recognize (hybridize to) as many of the different strains, subtypes etc. for a given target as possible. For example, the software tries to identify one set of primers and probe that recognizes every known INF-A sequence in the database (these sequences are in database as INCLUDE files) but will not recognize any other viruses, bacteria, etc. (these sequences are in the database but are tagged as EXCLUDE files). Scoring sets of primers and probes based on the number of native sequences recognized reflects both conservation and coverage but presents it in a more relevant and accurate manner.

For example, the nucleic acid probes and primers of the invention hybridize with more target nucleic acid variants than competitor probes and primers. For example, the Influenza A primer & probe set designed against the matrix protein gene (INFA-MP set) hybridizes with perfect complimentarity to 0.5484 (334 out of 609) matrix protein nucleic acid sequences variants identified within Genbank. This INFA-MP set will also hybridize with additional matrix protein sequence variants that are not identical.

Forward primer:

(SEQ ID NO:1)

5′-CTCATGGAATGGCTAAAGACAAGAC-3′
Probe:

(SEQ ID NO:2)

5′-AGTCCTCGCTCACTGGGCACGGT-3′
Reverse primer:

(SEQ ID NO:3)

5′-GGCATTTTGGACAAAGCGTCTAC-3′

By comparison, the Influenza A matrix protein gene primers & probes (SEQ ID Nos: 30, 32, and 34) described in U.S. Pat. No. 6,015,664 to Henrickson hybridize with perfect complimentarity to only 0.4351 (265 out of 609 matrix protein sequences identified within Genbank).

Primer ID #30 -

(SEQ ID NO:95)

CTTCTAACCGAGGTCGAAACGTA
Primer ID #34 -

(SEQ ID NO:96)

CGTCTACGCTGCAGTCCTCGCTCAC
Probe ID #32 -

(SEQ ID NO:97)

GGCTAAAGACAAGACCAATCCTGTCACCTCTGACTAA

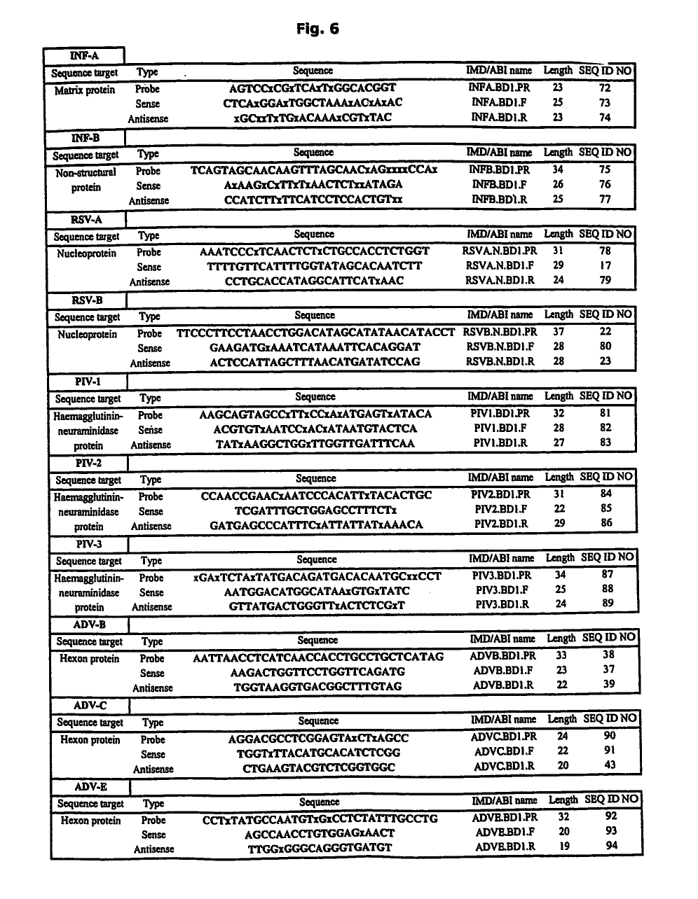

It is not always possible to identify a single primer/probe set that recognizes all the native target variants. Parameters are therefore chosen that identify primers and probes that recognize as close to 100% without compromising (a) the sequence's ability to perform PCR or (b) the sequence's specificity for recognizing just the native sequences. The ranking for specificity takes into account (i) how many degenerate bases are acceptable; (ii) where they occur, and (iii) a ranking of the native sequences that are identified or not identified by the primer/probe set. FIG. 6 illustrates degenerate bases in primers and probes, which are marked with an x. The term “degenerate” means a base position where two or more bases are known to occur in the native sequences. The phrase “ranking the native sequences” means weighting the annotations for each native sequence (e.g., strain type, country, year, etc).

Ranking begins by choosing the primer/probe set that recognized the most native sequences without any degenerate bases. The primer/probe sets are ranked according to (i) least number of degenerate bases (if more than one, they would not occur on the same primer or probe); (ii) location of the degenerate bases (e.g., not at the last 5 bases of 3′ end of the primers, not in the middle third of the probe). Anywhere else they would be weighted according to their position, for example—least important would be those degenerate bases closest to the 5′ end of the primer, next would be those closest to the 3′ end of the probe; next would be those closest to the 5′ end of the probe and (iii) the medical importance of native sequences are that are not identified by the candidate primer & probe set important.

If all of these parameters produce two or more primers/probe sets with identical abilities to recognize the native sequences, they are then ranked on their PCR penalty scores. The PCR parameters mentioned above will only be relaxed (e.g., longer amplicon) if (A) they do not generate any primer/probe sets or (B) the primer/probe sets recognize enough of the native sequences. If that fails two primers/probe sets or additional primers or probes can be used on the same target, where the combined sets will recognizes all the native sequences.

Sequence Selection and Classification

The relevant sequences of a particular target are collected and classified to determine which sequences should be the candidate for downstream primer design.

Alignment and Scoring

The target/native sequences of Step 1 are aligned, a consensus sequence is generated, and each base position in this sequences is scored according to percent identity, conservation, and coverage, to determine which regions of the consensus sequence should be targeted by the primers. In an embodiment, alignment of the sequences is done manually using the program ClustalW to align the sequences and the program GeneDoc to crop the aligned sequences to areas of interest or areas of maximum coverage. The PriMD™ software is then provided with the alignment file and it selects candidate primers and probes. The PriMD™ software then determines the identity, conservation, and coverage scores for each base of the candidate primers or probes. This information is then used to rank the sets of sequences. The PriMD™ software uses the same algorithm as Primer3 for selecting primers. TaqMan probes are selected using the criteria previously described by Holland, P. M., R. D. Abramson, R. Watson, and D. H. Gelfand. 1991. Proc. Natl. Acad. Sci. USA 88:7276-7280. The primer & probe sets are ranked according to a PCR penalty score. This PCR penalty, in turn, is one component of the PriMD™ software's overall ranking system.

Primer & Probe Design

This component of PriMD™ evaluates all possible primer and probe set possibilities and produces an exhaustive output of all valid primer sets. Primer sets are ranked according to many criteria, including (1) the ability to detect the target alignment sequences but not a set of exclude sequences; and (2) conformation to a particular DNA amplification technology, for example TaqMan® Real Time PCR. Other technologies include using Scorpion™ primers, Molecular Beacons, SimpleProbes, HyBeacons, Cycling Probe Technology, Invader Assay, Self-sustained Sequence Replication, Nucleic Acid Sequence-based Amplification, Ramification Amplifying Method, Hybridization Signal Amplification Method, Rolling Circle Amplification, Multiple Displacement Amplification, Thermophilic Strand Displacement Amplification, Transcription-mediated Amplification, Ligase Chain Reaction, Signal Mediated Amplification of RNA Technology, Split Promoter Amplification Reaction, Ligase Chain Reaction, Q-Beta Replicase, Isothermal Chain Reaction, One Cut Event Amplification System, Loop-mediated Isothermal Amplification, Molecular Inversion Probes, Ampliprobe, Headloop DNA amplification, Ligation Activated Transcription.

Ranking of Primer & Probe Sets

Valid primer & probe sets are ranked according to the criteria described above. PriMD may employ one or more metrics for a particular ranking. PriMD uses several methods to combine metrics, including:
1. Joint ranking—a single value is computed for the joint collection of metrics for each oligonucleotide;
2. Hierarchical ranking—oligonucleotide sets are sorted according to one metric, and each collection of oligonucleotide sets having the same ranking is then ranked further according to another metric. Several layers of hierarchical ranking may be used.
3. Serial ranking—all oligonucleotide sets are sorted according to a single metric, and the resultant ranking is then sorted according to another ranking in a manner that best conserves the first ranking. Multiple rankings may be used in succession.

In one ranking scheme, PriMD calculates each ranking in a uniform way, regardless of the type of ranking algorithm or metrics for the particular ranking. For a particular ranking, each oligo set is represented as a vector of quality metrics employed for that ranking. Each ranking is also assigned an ideal vector that represents the best values for each quality metric. Each component of the vector is assigned a default weight. The user may override these defaults by providing alternative weights. Next PriMD may normalize the vector data. PriMD then calculates a numerical value that measures the degree if dissimilarity of each oligonucleotide set vector from the ideal vector. Finally PriMD sorts the oligonucleotide sets according to this degree of dissimilarity. One method to determine a this degree of dissimilarity is to use the Euclidian distance function shown below:
D=sqrt(w₁(x₁−p₁)²+w₂(x₂−p₂)²+w₃(x₃−p₃)²+ . . . )
where: x₁ represents quality metric 1, x₂ represents quality metric 2, etc., w₁ represents the weight for metric 1, w₂ represents the weight for metric 2, etc., and p₁ represents the ideal value of metric 1, p₂ represents the ideal value of metric 2, etc.
PriMD™ Database

The PriMD™ database is a component of the PriMD™ system, which also includes the PriMD™ software. It is a central repository of all information used to run the PriMD™ software, as well as all data that went into making each primer/probe set. The database allows the user to log their processes and query their accumulating data. For example, the database allows the user to determine how up-to-date each oligonucleotide set is, in comparison to newer sequences. The database includes (1) Sequences (downloaded from Genbank, Influenza Sequence Database, etc.), including additional information described above; (2) Alignments (performed, e.g., by Clustal); (3) Commercial data (e.g., competitor's primers and probes, and our analysis of them); (4) Patents; (5) Data and results of each PriMD™ production run; and (6) Decisions and data for each final product.

Primers and Probes

The invention also provides nucleic acid primers, probes, primer sets, and primers/probe sets with substantial sequence identity to the nucleic acids disclosed herein, or the complement thereof. Thus, the invention provides nucleotide sequences having one or more nucleotide deletions, insertions, or substitutions relative to a nucleic acid sequence of any one of SEQ ID NOs: 1-94. The nucleic acids of the invention (e.g., RNA, DNA, PNA or chimeras) may be single-stranded, double stranded, or a mixed hybrid.

The invention also provides expression vectors, cell lines, and organisms comprising the nucleic acids. Some of the vectors, cells, or organisms are capable of expressing the encoded nucleic acids. Using the guidance of this disclosure, the nucleic acids of the invention can be produced by recombinant means. See, e.g., Sambrook et al. (1989) Molecular Cloning: A Laboratory Manual, 2nd Ed., Vols. 1-3, Cold Spring Harbor Laboratory; Berger and Kimmel (1987) Methods In Enzymology, Vol. 152: Guide To Molecular Cloning Techniques, San Diego Academic Press, Inc.; Ausubel et al. (1999) Current Protocols In Molecular Biology, Greene Publishing and Wiley-Interscience, New York. Alternatively, nucleic acids or fragments can be chemically synthesized using routine methods well known in the art (see, e.g., Narang et al. (1979) Meth. Enzymol. 68:90; Brown et al. (1979) Meth. Enzymol. 68:109; Beaucage et al. (1981) Tetra. Lett. 22:1859).

Some nucleic acids of the invention contain non-naturally occurring bases (e.g., deoxyinosine) or modified backbone residues or linkages that are prepared using methods as described in, e.g., Batzer et al. (1991) Nucleic Acid Res. 19:5081; Ohtsuka et al. (1985) J. Biol. Chem. 260:2605-2608; Rossolini et al. (1994) Mol. Cell. Probes 8:91-98. For example, the use of locked nucleic acids™, peptide nucleic acids, nucleotides containing inosine, methylated nucleotides, thio-phosphate nucleotides, aminoallyl modified nucleotides, Super G™ & Super N™ (Epoch Biosciences) are contemplated.

The invention provides nucleic acid probes and/or primers for detecting and/or amplifying target nucleic acids. Some of the nucleic acids comprise at least 10 contiguous bases identical or exactly complementary to any one of SEQ ID NOs: 1-94, usually at least about 10 bases, at least about 12 bases, at least about 14 bases, at least about 16 bases, at least about 18 bases, at least about 20 bases, at least about 22 bases, at least about 24 bases, at least about 26 bases, at least about 28 bases, at least about 30 bases, at least about 32 bases, at least about 34 bases, at least about 36 bases, or at least about 38. Some of the probes and primers having a sequence of one of SEQ ID NOs: 1-94, or a fragment thereof, are used in the methods (e.g., diagnostic methods) of the invention or in preparation of diagnostic compositions.

In an embodiment, the probes and primers are modified, e.g., by adding restriction sites to the probes or primers. In another embodiment, the primers or probes of the invention comprise additional sequences, such as linkers. The primer or probe sequences can also include nucleotide substitutions, additions, deletions, transitions, transpositions, or modifications, or other nucleic acid sequence alterations or non-nucleic acid moieties so long as specific binding to the relevant target nucleic acid corresponding to a target RNA or its gene is retained as a functional property of the polynucleotide.

In another embodiment, the primers or probes of the invention are modified with detectable labels. For example, the primers and probes are chemically modified, e.g., derivatized, incorporating modified nucleotide bases, or containing a ligand capable of being bound by an anti-ligand (e.g., biotin).

The primers of the invention can be used for a number of purposes, e.g., for amplifying a target nucleic acid in a biological sample for detection, or for cloning target genes from a variety of species. Using the guidance of the present disclosure, primers can be designed for amplification of a portion of a target nucleic acid gene or isolation of other target nucleic acid variants.

The nucleic acids of the invention (e.g., DNA, RNA, modifications, and analogues) can be made using any suitable method for producing a nucleic acid, such as the chemical synthesis and recombinant methods disclosed herein. Some nucleic acids of the invention are prepared by de novo chemical synthesis or by cloning. For example, a nucleic acid that hybridizes to a target nucleic acid can be made by inserting (ligating) a target DNA sequence (e.g., one of SEQ ID Nos: 1-94, or fragment thereof) in reverse orientation operably linked to a promoter in a vector (e.g., plasmid). Provided that the promoter and, preferably, termination and polyadenylation signals, are properly positioned, the strand of the inserted sequence corresponding to the non-coding strand will be transcribed and act as a primer or probe of the invention.

Probes

The TaqMan reaction consists of a pair of conventional PCR primers and a sequence-specific probe that binds to an internal region of the PCR product. The probe contains a fluorescent reporter dye on the 5′ base, and a quenching dye at the 3′ end. The dyes are chosen such that the emission of the reporter dye overlaps the absorbance of the quencher. The quencher can release the energy in the form of fluorescence at a different wavelength or in the form of heat. When illuminated the fluorescent energy of the reporter dye is effectively quenched as long as the two dyes remain in close proximity resulting in little or no detectable fluorescence. This is an example of fluorescent resonant energy transfer (FRET). The TaqMan assay exploits the endogenous 5′ nuclease activity of the DNA polymerase to liberate the fluorescent reporter in proportion to the amount of target. When the DNA polymerase replicates the target upon which a TaqMan probe is bound, its 5′ nuclease activity cleaves the probe thereby releasing the quencher and enabling the reporter dye to fluoresce. This dependence on polymerization ensures that cleavage of the probe occurs only if the target sequence is being amplified thus ignoring non-specific amplifications and primer oligomerization. This signal increases in direct proportion to the amount of PCR product in a reaction and is produced in real time.

Other examples of FRET probes consist of a pair of fluorescent probes that hybridize in close proximity on the target sequence. The donor probe is labeled with fluorophore at the 3′ end and the acceptor probe at 5′ end. During PCR, the two different oligonucleotides hybridize to adjacent regions of the target nucleic acid such that the fluorophores, which are coupled to the oligonucleotides, are in close proximity in the hybrid structure. The donor fluorophore is excited by an external light source, then passes part of its excitation energy to the adjacent acceptor fluorophore. The excited acceptor fluorophore emits light at a different wavelength which can then be detected and measured.

Another type of FRET probe uses a hairpin loop to modulate fluorescence. These molecular beacon probes are single stranded hairpin shaped oligonucleotide probes. One end of the beacon is tagged with a fluorophore, and the other one is tagged with a quencher. In the presence of a complementary target, the “stem” portion of the beacon separates so that the probe can hybridize to its target. In the absence of a complimentary target nucleic acid, the beacon remains closed and there is no significant fluorescence. When the beacon unfolds in the presence of the complementary target sequence, the fluorophore is no longer quenched, and the molecular beacon fluoresces.

Scorpion® primers are bi-functional, consisting of a primer covalently linked to a probe. The molecule also exploits FRET using a reporter fluorophore and a quencher fluorophore. In the absence of the target, the quencher absorbs the fluorescence emitted by the fluorophore. During the PCR reaction, the molecule hybridizes to the target resulting in separation of the fluorophore and the quencher resulting in increased fluorescence. The Scorpion® primer contains the probe element at the 5′ end. The probe is a self-complementary stem sequence with a fluorophore at one end and a quencher at the other. The primer sequence is modified at the 5′ end with a PCR blocker.

Other types of probes include: simple capture probes, designed for isolation methods and microarrays; melting-curve or end point probes, these are fluorescent probes which show marked increase in fluorescence when bound to their PCR target. (See http://www.european-patent-office.org/filingsoft/strand/table_a_b.htm).

Diagnostic Assays

The present methods provide means for determining if a subject has (diagnostic) or is at risk of developing (prognostic) a disease, condition or disorder that is associated with an aberrant target gene activity, e.g., an aberrant level of target DNA, RNA or protein, an aberrant bioactivity, or the presence of a mutation or particular polymorphic variant in the target gene.

Any body fluid, cell or tissue can be used to obtain nucleic acids for use in the diagnostic assays of the invention, such as, for example, blood, serum, plasma, sputum, urine, stool, skin, cerebrospinal fluid, saliva, gastric secretions, and tears. The tissue sample may be fresh, fixed, preserved, or frozen. Alternatively, nucleic acid tests can be performed on dry samples (e.g., hair or skin). For prenatal diagnosis, fetal nucleic acid samples can be obtained from maternal blood as described in WO91/07660. Alternatively, amniocytes or chorionic villi can be obtained for performing prenatal testing.

Diagnostic procedures can also be performed in situ directly on tissue sections (e.g., fresh, fixed, or frozen) of patient tissue obtained from biopsies or resections, such that no nucleic acid purification is necessary. Nucleic acid reagents can be used as probes and/or primers for such in situ procedures (see, e.g., van der Luijt et al. (1994) Genomics 20:1-4).

In certain embodiments of the invention, abnormal mRNA levels of target protein are detected by means such as Northern blot analysis, reverse transcription-polymerase chain reaction (RT-PCR), in situ hybridization, immunoprecipitation, Western blot hybridization, or immunohistochemistry, microarrays or combinations of above. In certain embodiments, cells are obtained from a subject and the target gene mRNA level is determined and compared to the level of target gene mRNA level in a healthy subject. An abnormal level of a target gene mRNA is likely to be indicative of an aberrant target gene activity.

In some methods, the presence of genetic alteration in at least one of the target genes is detected. The genetic alteration to be detected include, e.g., deletion, insertion, substitution of one or more nucleotides, a gross chromosomal rearrangement of a target gene, an alteration in the level of a messenger RNA transcript of a target gene, or inappropriate post-translational modification of a target gene polypeptide. The genetic alteration can be detected with various methods routinely performed in the art, such as sequence analysis, Southern blot hybridization, restriction enzyme site mapping, RFLP analysis and the like, and methods involving detection of the absence of nucleotide pairing between the nucleic acid to be analyzed and a probe. In such methods, polynucleotides isolated from a sample from a subject can be amplified first with an amplification procedure such as self sustained sequence replication (Guatelli et al. (1990), Proc. Natl. Acad. Sci. USA 87: 1874-1878); transcriptional amplification system (Kwoh et al. (1989), Proc. Natl. Acad. Sci. USA 86: 1173-1177); or Q-Beta Replicase (Lizardi et al. (1988), Bio/Technology 6: 1197).

In some methods, the alteration in a target gene is detected by mutation detection analysis using chips comprising oligonucleotides (“DNA probe arrays”) as described, e.g., in U.S. Pat. No. 6,905,816 to Jacobs and Cronin et al. (1996) Human Mut. 7: 244. Detection of the alteration can also utilize the probe/primer in a polymerase chain reaction (PCR). See U.S. Pat. No. 4,683,195; U.S. Pat. No. 4,683,202); Landegran et al. (1988), Science 241: 1077-1080; and Nakazawa et al. (1994), Proc. Natl. Acad. Sci. USA 91: 360-364). In some methods, the genetic alteration is detected by direct sequencing using various sequencing schemes including automated sequencing procedures such as sequencing by mass spectrometry (See, e.g., PCT publication WO 94/16101; Cohen et al. (1996) Adv. Chromatogr. 36:127-162; and Griffin et al. (1993) Appl. Biochem. Biotechnol. 38:147-159).

Specific diseases or disorders can be associated with specific allelic variants of polymorphic regions of certain target genes that do not necessarily encode a mutated protein. Thus, the presence of a specific allelic variant of a polymorphic region of a target gene, such as a single nucleotide polymorphism (“SNP”), in a subject can render the subject susceptible to developing a specific disease or disorder. Polymorphic regions in genes, e.g., target genes, can be identified, by determining the nucleotide sequence of genes in populations of individuals. If a polymorphic region, e.g., SNP is identified, then the link with a specific disease can be determined by studying specific populations of individuals, e.g., individuals that developed a specific disease.

The invention further provides kits for use in diagnostics or prognostic methods for diseases or conditions associated with abnormal target gene activity, or for determining which target gene therapeutic should be administered to a subject, for example, by detecting the presence of target gene mRNA or protein in a biological sample. The kit can detect abnormal levels or an abnormal activity of target protein, RNA or a degradation product of a target protein or RNA. Some of the kits detect autoantibodies against a target gene polypeptide.

The kits can contain at least one nucleic acid primer or probe. For example, some kits contain a labeled compound or agent capable of detecting target gene mRNA in a biological sample; means for determining the amount of target protein in the sample; and means for comparing the amount of target protein in the sample with a standard. The compound or agent can be packaged in a suitable container. The kit can further comprise instructions for using the kit to detect target gene mRNA or protein. Some kits contain one or more nucleic acid probes capable of hybridizing specifically to at least a portion of a target gene or allelic variant thereof, or mutated form thereof. Preferably the kit comprises at least one oligonucleotide primer capable of differentiating between a normal target gene and a target gene with one or more nucleotide differences.

Practice of the invention will be still more fully understood from the following examples, which are presented herein for illustration only and should not be construed as limiting the invention in any way.

### EXEMPLIFICATION

### Example 1

### Exemplary Primer and Probe Sets

The genomes of micro-organisms, such as viruses and bacteria, show considerable intra-species variations because of their large population size, high mutation rates, and short life cycles. For example, there are at least 2000 different strains or subtypes of human Influenza A available in Genbank. These genetic variations within a single species can be significant hurdles for any diagnostic test that uses nucleic acid as a target.

In an embodiment, the invention relates to nucleic acid sequences that are designed to amplify & detect any genetically-diverse group (e.g., strains, subtypes, serotypes, etc.) of a clinically important virus. Provided below are sets of nucleic acids comprising a forward primer, a reverse primer, and a probe sequence for exemplary viral targets, including influenza type A (INF-A), influenza type B (INF-B), respiratory syncytial virus type A (RSV-A), respiratory syncytial virus type B (RSV-B), parainfluenza type 1 (PIV-1), parainfluenza type 2 (PIV-2), parainfluenza type 3 (PIV-3), adenovirus type B (ADV-B), adenovirus type C (ADV-C), and adenovirus type E (ADV-E).

Each sequence is selected for its ability to function as a primer or as a probe for performing optimal PCR and for how well the sequence represents, or is conserved in, the target organism. The primers are designed to hybridize to complimentary sequences that are unique and highly conserved to the particular virus. In the presence of the target virus, the primers will anneal and amplify a sequence that can be recognized either by hybridization with a labeled probe or by molecular weight using conventional gel electrophoresis. If the target is RNA (e.g., the influenza viruses, the respiratory syncytial viruses, or the parainfluenza viruses) the amplification starts with the reverse transcription of the single-stranded viral RNA genome to form complimentary DNA (cDNA), followed by polymerase chain reaction (PCR) of the cDNA or genomic DNA (e.g., adenovirus). The probe sequence is designed to bind to an internal region of the amplified material or amplicon. The probe is labeled with various reporter molecules. The probes are compatible with conventional in situ hybridization, as fluorescent resonant energy transfer (FRET) probes, or as capture sequences for microarrays. In the examplary sequences shown below the probe used is a hydrolysis or TaqMan® variety.

These sequences are all derived from a consensus sequence generated from a multiple sequence alignment using ClustalW. The original sequences were obtained from Genbank or other publicly available databases.

The examples represent differences at the species level but PriMD™ can entertain any target down to any defined genetic difference. For example, if the target was strain e.g. H5N1, the primer & probe set can identify as many of the H5N1 sequences (INCLUDE files) but not any other strains (EXLUDE files).

In the following primer/probe set examples, the primer and probe sequences are also shown boxed within the amplicon sequence.

Influenza A set from the matrix protein gene
(INFA-MP set)
Forward primer:

(SEQ ID NO:1)

5′-CTCATGGAATGGCTAAAGACAAGAC-3′
Probe:

(SEQ ID NO:2)

5′-AGTCCTCGCTCACTGGGCACGGT-3′
Reverse primer:

(SEQ ID NO:3)

5′-GGCATTTTGGACAAAGCGTCTAC-3′

Amplicon sequence:

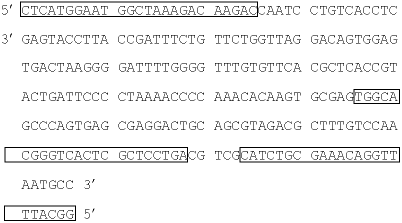

(SEQ ID NO:4)
Influenza B set from the non-structural protein gene (INFB-NS set)
Forward primer: 5′-ACAAGTCCTTATCAACTCTGCATAGA-3′ (SEQ ID NO:5)
Probe: 5′-TCAGTAGCAACAAGTTTAGCAACAAGCCTTCCAC-3′ (SEQ ID NO:6)
Reverse primer: 5′-CCATCTTCTTCATCCTCCACTGTAA-3′ (SEQ ID NO:7)
Amplicon sequence:

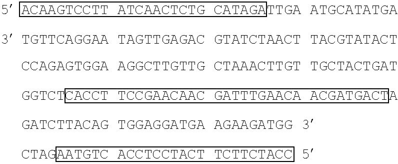

(SEQ ID NO:8)
Respiratory Syncytial Virus A Glycoprotein gene (RSVA-G set)
Forward primer: 5′-AGCAAGCCCACCACAAAACA-3′ (SEQ ID NO:9)
Probe: 5′-CGCCAAAACAAACCACCAAACAAACCCAA-3′ (SEQ ID NO:10)
Reverse primer: 5′-TGCAGGGTACAAAGTTGAACACT -3′ (SEQ ID NO:11)
Amplicon sequence:

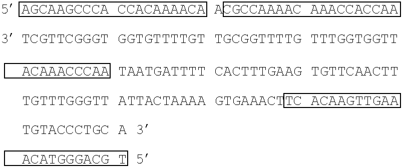

(SEQ ID NO:12)
Respiratory Syncytial Virus B Glycoprotein gene (RSVB-G set)
Forward primer: 5′-TCATAATTGCAGCCATAATATTCATCATC-3′ (SEQ ID NO:13)
Probe: 5′-TGCCAATCACAAAGTTACACTAACAACGGTCACA-3′ (SEQ ID NO:14)
Reverse primer: 5′-GCTAACCCTTTCTGGTGAGACTT-3′ (SEQ ID NO:15)
Amplicon sequence:

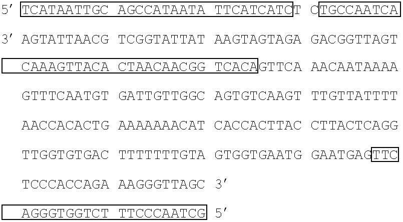

(SEQ ID NO:16)
Respiratory Syncytial Virus A Nucleocapsid gene (RSVA-N set)
Forward primer: 5′-TTTTGTTCATTTTGGTATAGCACAATCTT-3′ (SEQ ID NO:17)
Probe: 5′-AAATCCCTTCAACTCTACTGCCACCTCTGGT-3′ (SEQ ID NO:18)
Reverse primer: 5′-CCTGCACCATAGGCATTCATAAAC-3′ (SEQ ID NO:19)
Amplicon sequence:

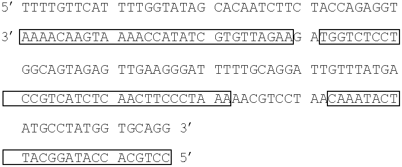

(SEQ ID NO:20)
Respiratory Syncytial Virus B Nucleocapsid gene (RSVB-N set)
Forward primer: 5′-GAAGATGCAAATCATAAATTCACAGGAT-3′ (SEQ ID NO:21)
Probe: 5′-TTCCCTTCCTAACCTGGACATAGCATATAACATACCT-3′ (SEQ ID NO:22)
Reverse primer: 5′-ACTCCATTAGCTTTAACATGATATCCAG-3′ (SEQ ID NO:23)
Amplicon sequence:

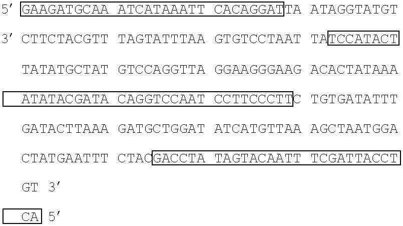

(SEQ ID NO:24)
Parainfluenza 1 HN gene (PIV1-HN set)
Forward primer: 5′ACGTGTTAATCCTACCATAATGTACTCA-3′ (SEQ ID NO:25)
Probe: 5′-AAGCAGTAGCCCTTCCCGAAATGAGTGATACA-3′ (SEQ ID NO:26)
Reverse primer: 5′-TATTAAGGCTGGTTTGGTTGATTTCAA-3′ (SEQ ID NO:27)
Amplicon sequence:

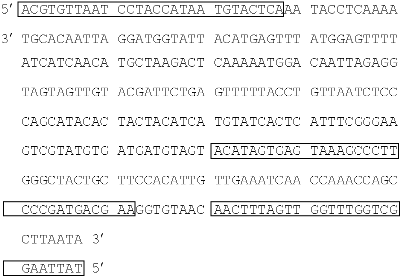

(SEQ ID NO:28)
Parainfluenza 2 HN gene (PIV12-HN set)
Forward primer: 5′-TCGATTTGCTGGAGCCTTTCTC-3′ (SEQ ID NO:29)
Probe: 5′-CCAACCGAACCAATCCCACATTCTACACTGC-3′ (SEQ ID NO:30)
Reverse primer: 5′-GATGAGCCCATTTCAATTATTATCAAACA-3′ (SEQ ID NO:31)
Amplicon sequence:

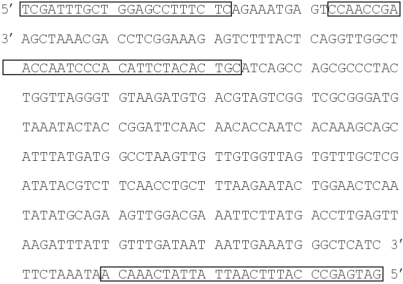

(SEQ ID NO:32)
Parainfluenza 3 HN gene (PIV3-HN set)
Forward primer: 5′-AATGGACATGGCATAATGTGCTATC-3′ (SEQ ID NO:33)
Probe: 5′-TGAGTCTAATATGACAGATGACACAATGCTCCCT-3′ (SEQ ID NO:34)
Reverse primer: 5′-GTTATGACTGGGTTCACTCTCGAT-3′ (SEQ ID NO:35)
Amplicon sequence:

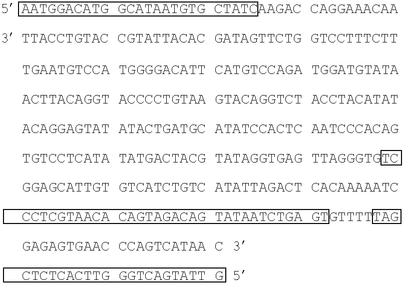

(SEQ ID NO:36)
Adenovirus-B Hexon gene (ADVB-H set)
Forward primer: 5′AAGACTGGTTCCTGGTTCAGATG-3′ (SEQ ID NO:37)
Probe: 5′-AATTAACCTCATCAACCACCTGCCTGCTCATAG-3′ (SEQ ID NO:38)
Reverse primer: 5′-TGGTAAGGTGACGGCTTTGTAG-3′ (SEQ ID NO:39)
Amplicon sequence:

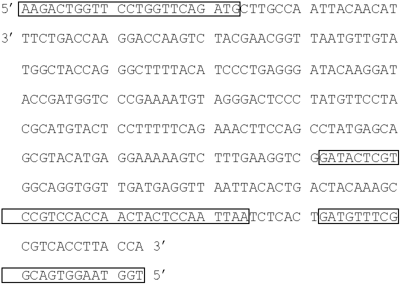

(SEQ ID NO:40)
Adenovirus-C Hexon gene (ADVC-H set)
Forward primer: 5′-TGGTCTTACATGCACATCTCGG-3′ (SEQ ID NO:41)
Probe: 5′-AGGACGCCTCGGAGTACCTGAGCC-3′ (SEQ ID NO:42)
Reverse primer: 5′-CTGAAGTACGTCTCGGTGGC-3′ (SEQ ID NO:43)
Amplicon sequence:

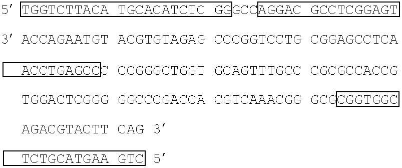

(SEQ ID NO:44)
Adenovirus-E Hexon gene (ADVE-H set)
Forward primer: 5′-AGCCAACCTGTGGAGGAACT-3′ (SEQ ID NO:45)
Probe: 5′-CCTCTATGCCAATGTTGCCCTCTATTTGCCTG-3′ (SEQ ID NO:46)
Reverse primer: 5′-TTGGTGGGCAGGGTGATGT-3′ (SEQ ID NO:47)
Amplicon sequence:

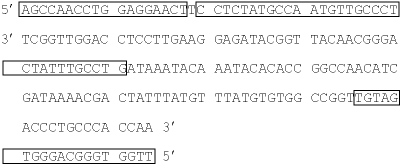

(SEQ ID NO:48)

### Example 2

### Exemplary Conserved Regions

The primers and probes in Example 1 are shown within the context of larger conserved regions of the genes. In some cases the primer or probe comprises the sequence of the complementary strand of the strand shown. The areas flanking the primers and probes provide additional sequence for candidate primers and probes.

Influenza A set from the matrix protein gene
(INFA-MP set)
For forward primer:

(SEQ ID NO:49)

5′ GATCTTGAGGCTCTCATGGAATGGCTAAAGACAAGACCAAT-3′
For reverse primer & probe (complimentary strand):

(SEQ ID NO:50)

5′TCGGCATTTTGGACAAAGCGTCTACGCTGCAGTCCTCGCTCACTGGGC
ACGGTGAGCGTGAA-3′
Influenza B set from the non-structural protein
gene (INFB-NS set)
Forward primer, probe, and reverse amplicon:

(SEQ ID NO:51)

5′ AATGGATACAAGTCCTTATCAACTCTGCATAGATTGAATGCATATGA
CCAGAGTGGAAGGCTTGTTGCTAAACTTGTTGCTACTGATGATCTTACAG
TGGAGGATGAAGAAGATGGCCATCGGATCCTCAA-3′
Respiratory Syncytial Virus A Glycoprotein gene
(RSVA-G set)
Forward primer & probe:

(SEQ ID NO:52)

5′AGCAAGCCCACCACAAAACAACGCCAAAACAAACCACCAAACAAACCC
AA-3′
For reverse primer (complimentary strand):

(SEQ ID NO:53)

5′GTTGGATTGTTGCTGCATATGCTGCAGGGTACAAAGTTGAACACTTCA
AAGTGAAAAT-3′
Respiratory Syncytial Virus B Glycoprotein gene
(RSVB-G set)
Forward primer & probe:

(SEQ ID NO:54)

5′TTTTGGCAATGATAATCTCAACCTCTCTCATAATTGCAGCCATAATAT
TCATCATCATCTCTGCCAATCACAAAGTTACACTAACAACGGTCACA GT
T-3′
Reverse primer (complimentary strand):

(SEQ ID NO:55)

5′GGTTGTTTGGATGGGCTAACCCTTTCTGGTGAGACTTGAGTAAGGTAA
GTGGTGATGTTTTT-3′
Respiratory Syncytial Virus A Nucleocapsid gene
(RSVA-N set)
Forward primer, probe, and reverse amplicon:

(SEQ ID NO:56)

5′CACTTTATAGATGTTTTTGTTCATTTTGGTATAGCACAATCTTCTACC
AGAGGTGGCAGTAGAGTTGAAGGGATTTTTGCAGGATTGTTTATGAATGC
CTATGGTGCAGGGCAAGTGATG
Respiratory Syncytial Virus B Nucleocapsid gene
(RSVB-N set)
Forward primer & probe:

(SEQ ID NO:57)

5′AACAAACTATGTGGTATGCTATTAATCACTGAAGATGCAAATCATAAA
TTCACAGGATTAATAGGTATGTTATATGCTATGTCCAGGTTAGGAAGGGA
AGA-3′
Reverse primer (complimentary strand):

(SEQ ID NO:58)

5′TTGACGATATGTTGTTATATCTACTCCATTAGCTTTAACATGATATCC
AGCATCTTTAAGTATCTTTATAG-3′
Parainfluenza 1 HN gene (PIV1-HN set)
Forward primer:

(SEQ ID NO:59)

5′ACATCACGTGTTAATCCTACCATAATGTACTCAA-3′
For reverse primer & probe (complimentary strand):

(SEQ ID NO:60)

5′ACTTGTCTTGAACAACATAGGTTGTAAGGTATTAAGGCTGGTTTGGTT
GATTTCAACAATGTGGAAGCAGTAGCCCTTCCCGAAATGAGTGATACATG
ATGTAGT-3′
Parainfluenza 2 HN gene (PIV12-HN set)
Forward primer:

(SEQ ID NO:61)

5′CCCAACTATCGATTTGCTGGAGCCTTTCTC-3′
Probe (sense strand):

(SEQ ID NO:62)

5′AAATGAGTCCAACCGAACCAATCCCACATTCTACACTGCATC-3′
Reverse primer (complimentary strand):

(SEQ ID NO:63)

5′AAATGGTATTATTTGGAACTCCCCTAAAAGAGATGAGCCCATTTCAAT
TATTATCAAACAATAAAT-3′
Parainfluenza 3 HN gene (PIV3-HN set)
Forward primer:

(SEQ ID NO:64)

5′ATAAAATGGACATGGCATAATGTGCTATCAAGACCAGGAAAC-3′
Probe (complimentary strand):

(SEQ ID NO:65)

5′TGAGTCTAATATGACAGATGACACAATGCTCCCTGT-3′
Reverse primer (complimentary strand):

(SEQ ID NO:66)

5′TGTTGAGTAAGTTATGACTGGGTTCACTCTCGATTT-3′
Adenovirus-B Hexon gene (ADVB-H set)
Forward primer:

(SEQ ID NO:67)

5′AACATGACCAAAGACTGGTTCCTGGTTCAGATGCTTGCCAA-3′
Probe & reverse primer (complimentary strand):

(SEQ ID NO68:)

5′ATTGGTAAGGTGACGGCTTTGTAGTCAGTGTAATTAACCTCATCAACC
ACCTGCCTGCTCATAGGCTGGAAGTTTCTGAAAAAGGAGTACATGCGA
T-3′
Adenovirus-C Hexon gene (ADVC-H set)
Forward primer & probe:

(SEQ ID NO:69)

5′ATGGCTACCCCTTCGATGATGCCGCAGTGGTCTTACATGCACATCTCG
GGCCAGGACGCCTCGGAGTACCTGAGCCCCCGGGCTGGTGCAGTT-3′
Reverse primer (complimentary strand):

(SEQ ID NO:70)

5′GCCACCGTGGGGTTTCTAAACTTGTTATTCAGGCTGAAGTACGTCTCG
GTGGC-3′
Adenovirus-E Hexon gene (ADVE-H set)
Forward primer, probe, and reverse primer:

(SEQ ID NO:71)

5′ACATCCAAGCCAACCTGTGGAGGAACTTCCTCTATGCCAATGTTGCCC
TCTATTTGCCTGATAAATACAAATACACACCGGCCAACATCACCCTGCCC
ACCAACACCAACACCTACGAGTACATGAA

### Example 3

### Exemplary Consensus Sequences

Variants of the nucleic acids described in Example 1 were aligned and consensus sequences were identified (FIG. 6). The symbol “x” indicates that the base was degenerate or variable, and therefore represents any nucleotide, e.g., A, G, C, T, or U, or functional equivalent thereof.

### INCORPORATION BY REFERENCE

The contents of all cited references (including literature references, patents, patent applications, and websites) that maybe cited throughout this application are hereby expressly incorporated by reference. The practice of the present invention will employ, unless otherwise indicated, conventional techniques of nucleic acid technology, software technology, and computer technology, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

We claim:

1. A polynucleotide for detecting an influenza virus type A nucleic acid, the polynucleotide comprising a sequence that shares at least about 70% identity with the sequence of SEQ ID NO: 1, or complement thereof.

2. A polynucleotide for detecting an influenza virus type A nucleic acid, the polynucleotide comprising a sequence that shares at least about 70% identity with the sequence of SEQ ID NO: 2, or complement thereof.

3. A polynucleotide for detecting an influenza virus type A nucleic acid, wherein the polynucleotide hybridizes to a nucleic acid comprising the sequence of SEQ ID NO: 1, or complement thereof.

4. A polynucleotide for detecting an influenza virus type A nucleic acid, wherein the polynucleotide hybridizes to a nucleic acid comprising the sequence of SEQ ID NO: 2, or complement thereof.

5. A polynucleotide for detecting an influenza virus type A nucleic acid, the polynucleotide comprising a sequence that shares at least about 70% identity with the sequence of SEQ ID NO: 3, or complement thereof.

6. A polynucleotide for detecting an influenza virus type A nucleic acid, wherein the polynucleotide hybridizes to a nucleic acid comprising the sequence of SEQ ID NO: 3, or complement thereof.

7. An polynucleotide for detecting an influenza virus type A nucleic acid, wherein the polynucleotide comprises the sequence CTCAxGGAxTGGCTAAAxACxAxAC (SEQ ID NO: 73), or complement thereof.

8. A polynucleotide for detecting an influenza virus type A nucleic acid, wherein the polynucleotide comprises the sequence xGCxxTxTGxACAAAxCGTxTAC (SEQ ID NO: 74), or complement thereof.

9. A polynucleotide set for detecting an influenza virus type A nucleic acid, wherein the polynucleotide set comprises the sequence CTCAxGGAxTGGCTAAAxACxAxAC (SEQ ID NO: 73), or complement thereof, and xGCxxTxTGxACAAAxCGTxTAC (SEQ ID NO: 74), or complement thereof.

10. The polynucleotide of claim 2, wherein the influenza virus type A nucleic acid is an amplification product.

11. The polynucleotide of claim 2, wherein the polynucleotide further comprising a label.

12. The polynucleotide of claim 11, wherein the label is a fluorescence energy transfer donor.

13. The polynucleotide of claim 2, wherein the polynucleotide is attached to a solid support.

14. The polynucleotide of claim 13, wherein the solid support is a microarray.

15. The polynucleotide of claim 2, wherein the polynucleotide is a hydrolysis probe.

16. A primer pair for amplifying an influenza virus type A nucleic acid, the primer pair comprising a first primer and a second primer, wherein the first primer comprises a sequence that shares at least about 70% identity with the sequence of SEQ ID NO: 1, or complement thereof, and wherein the second primer comprises a sequence that shares at least about 70% sequence identity with the sequence of SEQ ID NO: 3, or complement thereof.

17. A primer pair for amplifying an influenza virus type A nucleic acid, the primer pair comprising a first primer and a second primer, wherein the first primer hybridizes to a nucleic acid comprising the sequence of SEQ ID NO: 1, or complement thereof, and wherein the second primer hybridizes to a nucleic acid comprising the sequence of SEQ ID NO: 3, or complement thereof.

18. A primer pair for amplifying an influenza virus type A nucleic acid, the primer pair comprising a first primer and a second primer, wherein the first primer comprises the sequence of SEQ ID NO: 73, or complement thereof, and the sequence or SEQ ID NO: 74, or complement thereof.

19. A method for amplifying an influenza virus type A nucleic acid, the method comprising the step of:
amplifying a fragment of an influenza virus type A nucleic acid using a primer pair comprising a first primer and a second primer, wherein the first primer comprises a sequence that shares at least about 70% identity with the sequence of SEQ ID NO: 1, or complement thereof, and wherein the second primer comprises a sequence that shares at least about 70% identity with the sequence of SEQ ID NO: 3, or complement thereof.

20. A method for determining the presence or absence of an influenza virus type A nucleic acid in a sample, the method comprising the steps of:
(a) amplifying from a sample a fragment of an influenza virus type A nucleic acid using a primer pair comprising a first primer and a second primer, wherein the first primer comprises a sequence that shares at least about 70% identity with the sequence of SEQ ID NO: 1, or complement thereof, and wherein the second primer comprises a sequence that shares at least about 70% identity with the sequence of SEQ ID NO: 3, or complement thereof, and
(b) detecting the amplification product.

21. The method of claim 20, wherein the sample comprises a tissue sample.

22. The method of claim 21, wherein the tissue sample is selected from the group consisting of blood, serum, plasma, sputum, urine, stool, skin, cerebrospinal fluid, saliva, gastric secretions, tears, oropharyngeal swabs, nasopharyngeal swabs, throat swabs, nasal aspirates, nasal wash, and fluids collected from the ear, eye, mouth, and respiratory airways.

23. The method of claim 21, wherein the tissue sample is fixed or frozen.

24. The method of claim 19 or 20, wherein the nucleic acid comprises RNA.

25. The method of claim 19 or 20, wherein the nucleic acid comprises DNA.

26. The method of claim 19 or 20, wherein the amplifying step comprises polymerase chain reaction.

27. The method of claim 19 or 20, wherein the amplifying step comprises a TaqMan reaction.

28. The method of claim 19 or 20, wherein the amplifying step comprises isothermal amplification.

29. The method of claim 19 or 20, wherein the amplifying step is conducted on an array.

30. The method of claim 19 or 20, wherein the amplifying step comprises in situ hybridization.

31. The method of claim 20, wherein the detecting step comprises gel electrophoresis.

32. The method of claim 20, wherein the detecting step comprises hybridization to a labeled probe.

33. The method of claim 20, wherein the label is selected from the group consisting of biotin, at least one fluorescent moiety, an antigen, a molecular weight tag, and a modifier of probe Tm.

34. The method of claim 20, wherein the detecting step comprises in situ hybridization.

35. The method of claim 20, wherein the detecting step comprises fluorescence resonant energy transfer (FRET).

36. The method of claim 20, wherein the detecting step comprises measuring fluorescence.

37. The method of claim 20, wherein the detecting step comprises measuring mass.

38. The method of claim 20, wherein the detecting step comprises measuring charge.

39. The method of claim 20, wherein the detecting step comprises measuring chemiluminescence.

40. A method for designing a probe for identifying a plurality of nucleic acid variants, the method comprising the steps of:
(a) identifying nucleotide identities between at least two nucleic acid sequences that are representative of at least two target variants;
(b) selecting at least two candidate probe sequences that define a probe that can hybridize with the at least two nucleic acid sequences; and
(c) ranking the probe sequences according to the percentage identity to the nucleic acid sequences, thereby determining an optimal probe sequence for identifying a plurality of target variants.

41. A method for designing a probe for identifying a plurality of marker variants, the method comprising the steps of:
(a) identifying nucleotide identities between at least two nucleic acid sequences that are representative of at least two target variants;
(b) selecting at least two candidate probe sequences that define a probe that can hybridize with the at least two nucleic acid sequences; and
(c) ranking the probe sequences according to conservation scores for the probe sequences, thereby determining an optimal probe sequence for identifying a plurality of target variants.

42. A method for designing a primer for synthesizing a nucleic acid strand in a plurality of marker variants, the method comprising the steps of:
(a) identifying nucleotide identities between at least two nucleic acid sequences that are representative of at least two target variants;
(b) selecting at least two candidate primer sequences that define a primer that can hybridize with the at least two nucleic acid sequences; and
(c) ranking the primer sequences according to the percentage identity to the nucleic acid sequences, thereby determining an optimal primer sequence for identifying a plurality of target variants.

43. A method for designing a primer pair for amplifying a nucleic acid in a plurality of marker variants, the method comprising the steps of:
(a) identifying nucleotide identities between at least two nucleic acid sequences that are representative of at least two target variants;
(b) selecting at least two candidate forward primer sequences that define a forward primer that can hybridize with the at least two nucleic acid sequences;
(c) selecting at least two candidate reverse primer sequences that define a reverse primer that can hybridize with the at least two nucleic acid sequences;
(d) ranking the forward primer sequences according to the percentage identity to the nucleic acid sequences, thereby determining an optimal forward primer sequence for identifying a plurality of target variants; and
(e) ranking the reverse primer sequences according to the percentage identity to the nucleic acid sequences, thereby determining an optimal reverse primer sequence for identifying a plurality of target variants.

44. A method for designing a primer pair for amplifying a nucleic acid in a plurality of target variants and a probe for detecting an amplicon generated thereby, the method comprising the steps of:
(a) identifying nucleotide identities between at least two nucleic acid sequences that are representative of at least two target variants;
(b) selecting at least two candidate forward primer sequences that define a forward primer that can hybridize with the at least two nucleic acid sequences;
(c) selecting at least two candidate reverse primer sequences that define a reverse primer that can hybridize with the at least two nucleic acid sequences;
(d) selecting at least two candidate probe sequences that define a probe that can hybridize with the at least two nucleic acid sequences;
(e) ranking the forward primer sequences according to the percentage identity to the nucleic acid sequences, thereby determining an optimal forward primer sequence for identifying a plurality of target variants;
(f) ranking the reverse primer sequences according to the percentage identity to the nucleic acid sequences, thereby determining an optimal reverse primer sequence for identifying a plurality of target variants; and
(g) ranking the probe sequences according to the percentage identity to the nucleic acid sequences, thereby determining an optimal probe sequence for identifying a plurality of target variants.

45. The method according to any of claims 40-44, further comprising at least one of the steps selected from the group consisting of (i) determining a target sequence score for the candidate sequences; (ii) determining a mean conservation score for the candidate sequence(s); (iii) determining a mean coverage score for the candidate sequences; (iv) determining 100% conservation of a portion of the candidate sequence(s); (v) determining a species score (vi) determining a strain score; (vii) determining a subtype score; (viii) determining a serotype score; (ix) determining an associated disease score; (x) determining a year score; (xi) determining a country of origin score; (xii) determining a duplicate score; (xiii) determining a patent score; and (xiv) minimum qualifying score.

46. The method according to claim 45, wherein the portion is located at about the center of the sequence.

47. The method according to claim 45, wherein the portion is located at about the 5′ end of the sequence.

48. The method according to claim 45, wherein the portion is located at about the 3′ end of the sequence.

49. The method according to any one of claims 40-44, further comprising the step of allowing for one or more nucleotide changes when determining identity between the candidate sequences and the nucleic acid sequences.

50. The method according to any one of claims 40-44, further comprising the step of comparing the candidate sequences to exclusion sequences and rejecting those candidate sequences as optimal if they share identity with the exclusion sequences.

51. The method according to any one of claims 40-44, further comprising the step of comparing the candidate sequences to inclusion sequences and rejecting those candidate sequences as optimal if they do not share identity with the inclusion sequences.

52. The method according to any one of claims 40-44, wherein the nucleic acid sequences are representative of an infectious agent.

53. The method according to claim 52, wherein the infectious agent is selected from the group consisting of a virus, a bacteria, a fungus, and a parasite.

54. The method according to any one of claims 40-44, wherein the target is a disease marker.

55. The method according to any one of claims 40-44, wherein the target is a genetic marker.

56. The method according to any one of claims 40-44, wherein the target comprises an infectious agent that comprises at least two different kingdoms, phyla, classes, orders, families, genera, species, subtypes, and genotypes.

57. The method according to any one of claims 40-44, wherein the target comprises a number of serotypes or phenotypes.

58. The method according to any one of claims 40-44, wherein the target comprises a marker for drug resistance or drug susceptibility.

59. The method according to any one of claims 40-44, wherein the identifying step (a) comprises aligning the nucleic acid sequences.

60. The method according to any one of claims 40-44, wherein the identifying step (a) comprises a manual alignment of nucleic acid sequences in from database.

61. The method according to any one of claims 40-44, wherein the alignment is performed using a program selected from the group consisting of ClustalW, ClustalX, PileUp (GCG), MULTALIGN, and Tcoffee.

62. The method according to any one of claims 40-44, wherein the alignment is performed using a sum of pairs scoring method and/or optimization using an evolutionary tree.

63. The method according to any one of claims 40-44, wherein the alignment is performed using DNAStar's Lasergene.

64. The method according to any one of claims 40-44, wherein the database is an annotated database.

65. The method according to any one of claims 40-44, wherein the database is a PriMD™ database.

66. The method according to any one of claims 40-44, wherein the database is selected from the group consisting of the Influenza Sequence Database, the Ribosomal Database, and Genbank database.

67. The method according to any one of claims 40-44, wherein the identifying step (a) comprises a BLAST analysis.

68. The method according to any one of claims 40-44, wherein the identifying step (a) further comprises the step of editing the alignment by removing at least one 5′ nucleotide and/or at least one 3′ nucleotide from at least one nucleic acid sequence.

69. The method according to any one of claims 40-44, wherein the identifying step (a) further comprises the step of editing the alignment by removing nucleic acid sequences that do not align.

70. The method according to claim 68, wherein the alignment is repeated after the editing step.

71. The method according to any one of claims 40-44, wherein the selecting step (b) comprises using a polymerase chain reaction penalty score formula.

72. The method according to claim 71, wherein the polymerase chain reaction penalty score formula comprises at least one of a weighted sum of difference between primer Tm and optimal Tm, difference between the primer Tms, amplicon length and distance between the primer and a TaqMan probe.

73. The method according to any one of claims 40-44, wherein the first selecting step (d) comprises determining which sequences or sets of sequences have mean conservation scores closest to 1.

74. The method according to claim 73, wherein a standard of deviation on the mean conservation scores for each sequence is compared.

75. The method according to any one of claims 40-44, wherein the first determining step comprises determining which sequences hybridize to the most target sequences.

76. The method according to any one of claims 40-44, wherein the ability of the candidate sequence to hybridize with a nucleic acid sequence of the most infectious agents is determined.

77. The method according to any one of claims 43-44, further comprising the step of evaluating which infectious agent sequences are hybridized by the optimal forward primer and optimal reverse primer.

78. The method according to claim 77, wherein the evaluating step comprises determining the number of base differences between nucleic acid sequences in a database.

79. The method according to claim 78, wherein a public database is used.

80. The method according to claim 78, wherein a PriMD™ database is used.

81. The method according to claim 77, wherein the evaluating step comprises performing an in silico polymerase chain reaction.

82. The method according to claim 77, wherein the evaluation step comprises rejecting the forward primer and reverse primer if it does not meet inclusion or exclusion criteria.

83. The method according to claim 77, wherein the evaluation step comprises rejecting the forward primer and reverse primer if it does not amplify a medically valuable nucleic acid.

84. The method according to claim 77, wherein the evaluation step comprises conducting a BLAST analysis to identify forward primer sequences and reverse primer sequences that overlap with a published and/or patented sequence.

85. The method according to claim 77, wherein the evaluation step comprises determining secondary structure of the forward primer sequence and/or the reverse primer sequence.

86. The method according to claim 77, wherein the secondary structure of the probe sequence and/or the target sequence is determined.

87. The method according to claim 77, further comprising the step of evaluating whether the forward primer sequence, reverse primer sequence, and/or probe sequence hybridizes to sequences in the database other than the nucleic acid sequences that are representative of the target variants.

88. A method for screening a sample for the presence or absence of a nucleic acid indicative of a disease, the method comprising the steps of:
(a) identifying at least one optimal primer or optimal probe capable of hybridizing to a nucleic acid indicative of a disease, according to the methods of any one of claims 40-45; and
(b) exposing the sample to the optimal primer or optimal probe under suitable hybridization conditions such that the optimal primer or optimal probe hybridizes to the nucleic acid if present in the sample; and
(c) detecting a hybridization reaction.

89. The method according to claim 88, wherein the sample comprises a tissue sample.

90. The method according to claim 89, wherein the tissue sample is selected from the group consisting of blood, serum, plasma, sputum, urine, stool, cells, skin, cerebrospinal fluid, saliva, gastric secretions, tears, oropharyngeal swabs, nasopharyngeal swabs, throat swabs, nasal aspirates, nasal wash, and fluids collected from the ear, eye, mouth and respiratory airways.

91. The method according to claim 88, wherein the nucleic acid comprises RNA.

92. The method according to claim 88, wherein the nucleic acid comprises DNA.

93. The method according to claim 88, wherein the detecting step comprises polymerase chain reaction.

94. The method according to claim 88, wherein the detecting step comprises a TaqMan reaction.

95. The method according to claim 88, wherein the detecting step comprises isothermal amplification.

96. The method according to claim 88, wherein the detecting step is conducted on an array.

97. The method according to claim 88, wherein the detecting step comprises in situ hybridization.

98. The method according to claim 88, wherein the detecting step comprises gel electrophoresis.

99. The method according to claim 88, wherein the detecting step comprises hybridization to a probe comprising a label.

100. The method according to claim 99, wherein the label is selected from the group consisting of biotin, at least one fluorescent moiety, an antigen, a molecular weight tag, and a modifier of Tm.

101. The method according to claim 89, wherein the detecting step comprises fluorescence resonant energy transfer.

102. The method according to claim 89, wherein the detecting step comprises measuring fluorescence.

103. The method according to claim 89, wherein the detecting step comprises measuring mass.

104. The method according to claim 89, wherein the detecting step comprises measuring charge.

105. The method according to claim 89, wherein the detecting step comprises measuring chemiluminescence.

106. A method for designing a primer pair for amplifying a nucleic acid in a plurality of target variants and a probe for detecting an amplicon generated thereby, the method comprising the steps of:
(a) identifying nucleotide identities between at least two nucleic acid sequences that are representative of at least two target variants;
(b) selecting at least one candidate forward primer sequence that defines a forward primer that can hybridize with the at least two nucleic acid sequences;
(c) selecting at least one candidate reverse primer sequence that defines a reverse primer that can hybridize with the at least two nucleic acid sequences;
(d) selecting at least one candidate probe sequence that define a probe that can hybridize with the at least two nucleic acid sequences;
(e) ranking the forward primer, reverse primer, and probe sequences according to percentage identity to the nucleic acid sequences, thereby determining an optimal primer/probe set for identifying a plurality of target variants, the set comprising a forward primer, a reverse primer, and a probe sequence.

107. A computer-implemented system for identifying oligonucleotides for detecting multiple variants of a target, comprising:
a user interface for specifying a target;
software for reading a multiple alignment of nucleic acid sequences for a plurality of variants of the target;
software for generating a representative sequence based at least in part upon the multiple alignment; software for computing a plurality of oligonucleotides that are complementary to portions of the representative sequence; and
software for assigning a quality metric to each computed oligonucleotide responsive to an extent to which the respective oligonucleotide aligns with each of the variants of the target.

108. A computer-implemented system as recited in claim 107, further comprising:
software for organizing the computed oligonucleotides into sets; and
software for assigning a quality metric to each set responsive to an extent to which the oligonucleotides in the respective set together are able to detect variants of the target using a predetermined detection/amplification technology.

109. A computer-implemented system as recited in claim 107, further comprising:
software for assigning a quality metric to each oligonucleotide responsive to any of—
its patent novelty,
any strain that the oligonucleotide can detect,
year that any strain that the oligonucleotide can detect was isolated,
region of geographical prevalence of the strain,
medical need of patients infected by the strain,
any disease associated with the oligonucleotide, and
treatability of any disease associated with the oligonucleotide.

110. A computer-implemented system, comprising:
software for computing a plurality of oligonucleotide sets for detecting multiple variants of a target;
software for assigning at least one quality metric to each of the plurality of oligonucleotide sets; and
software for ranking the plurality of oligonucleotide sets responsive to the at least one quality metric.

111. A computer-implemented system as recited in claim 110, wherein the software for ranking comprises a mathematical function or algorithm operative in response to the at least one quality metric.

112. A computer-implemented system as recited in claim 111, wherein the at least one quality metric is a plurality of quality metrics, and the function or algorithm further comprises software for weighting different quality metrics differently.

113. A computer-implemented system as recited in claim 111, wherein the mathematical function or algorithm is arranged for computing a degree of dissimilarity between each at least one quality metric and an ideal value for each at least one quality metric.

114. A computer-implemented system as recited in claim 113, wherein the degree of dissimilarity can be expressed as a distance D,
wherein D=sqrt(w₁(x₁−p₁)²+w₂(x₂−p₂)²+w₃(x₃−p₃)²+ . . . ),
wherein wᵢ is a weight given to the i^{th} quality metric, xᵢ is a score given for the i^{th} metric, and pᵢ is a perfect score for the i^{th} metric.

115. A computer-implemented system as recited in claim 110, wherein the software for ranking performs any of a joint ranking, a hierarchical ranking, and a serial ranking of the plurality of oligonucleotide sets.

116. A computer-implemented system as recited in claim 110, wherein the at least one quality metric is a plurality of quality metrics, and the software for ranking is user controllable for generating a plurality of rankings responsive to different groupings of quality metrics.

117. A computer-implemented system for identifying oligonucleotide sets for detecting target nucleic acids, comprising: a user interface for specifying a target;
a data collection for storing a plurality of data, including—
nucleic acid sequences for a plurality of known targets,
oligonucleotide sets corresponding to the nucleic acid sequences, or complements thereof, and
additional data, comprising at least one of alignment data, demographic data, patent data, and commercial data;
software for identifying any oligonucleotide sets in the data collection that are candidates for detecting the specified target nucleic acid; and
software for computing at least one quality metric for each identified oligonucleotide set responsive to any of the additional data stored in the data collection.

118. A computer-implemented system as recited in claim 117, wherein the software for computing the at least one quality metric for each identified oligonucleotide further comprises software for ranking all identified oligonucleotides responsive to the at least one quality metric.

119. A computer-implemented system for identifying oligonucleotide sets for detecting target nucleic acids, comprising:
a user interface for specifying a target;
a data collection for storing a plurality of data including oligonucleotide sets corresponding to a plurality of known targets;
software for identifying any oligonucleotide sets in the data collection that are candidates for detecting the specified target; and
a plurality of quality metrics for scoring each identified oligonucleotide set, wherein each quality metric is assigned a default weight and wherein the weight of each quality metric is adjustable via the user interface.

120. A computer-implemented system as recited in claim 119, wherein at least one of the plurality of quality metrics relates to alignment of a respective oligonucleotide set to the specified target.

121. A computer-implemented system as recited in claim 119, wherein the plurality of quality metrics comprises at least one metric related to suitability for a particular amplification and/or detection technology.

122. A computer-implemented system as recited in claim 121, wherein the at least one metric related to suitability comprises any of—
a difference between Tm and Opt™,
a difference between primer TMS,
amplicon length,
a distance between primer and probe,
PCR score, and
quality of hybridization.

123. A computer-implemented system as recited in claim 119 wherein the plurality of quality metrics comprises at least one metric related to alignment.

124. A computer-implemented system as recited in claim 123, wherein the at least one metric related to alignment comprises any of—
conservation,
coverage,
a degree of mismatch with the oligonucleotides of the set;
ISI-N family of scores that measure a fraction of target sequences that exhibit up to N mismatches to the oligonucleotides of the set,
a fraction of bases out of all possible bases that exhibit a mismatch to the oligonucleotides of the set,
a minimum number of allowable mismatches to identify all possible target sequences,
a quality of hybridization, and
medical need for detecting the target sequence.

125. A data collection, comprising:
nucleic acid sequences for a plurality of variants of a target; and
a multiple alignment of the nucleic acid sequences for the plurality of variants of the target.

126. A data collection as recited in claim 125, further comprising any of—
conservation data indicative of a degree of conservation among the plurality of variants, and
coverage data indicative of a degree of coverage among the plurality of variants.

127. A data collection as recited in claim 125, further comprising a consensus sequence of the multiple alignment.

128. A data collection as recited in claim 125, further comprising a plurality of oligonucleotide sequences that are candidates for binding with the nucleic acid sequences.

129. A data collection as recited in claim 128, further comprising at least one measure of suitability of each oligonucleotide sequence for binding with the nucleic acid sequences.

130. A data collection as recited in claim 128, further comprising at least one measure of suitability of each oligonucleotide sequence for use with a predetermined amplification and/or detection technology.

131. A data collection as recited in claim 130, wherein the predetermined amplification and/or detection technology is Taqman.

132. A data collection as recited in claim 128, further comprising, for each oligonucleotide sequence, at least one measure of any of—
patent novelty,
any strain that the oligonucleotide sequence can detect,
age of the strain,
region of geographical prevalence of the strain, and
medical need of organisms infected by the strain.

133. A data collection as recited in claim 128, further comprising data related to commercially available primers and probes.

134. A data collection as recited in claim 125, wherein the multiple alignment is an output of a computer program.

135. A data collection as recited in claim 125, further comprising nucleic acid sequences for a plurality of different targets, including variants thereof.

136. A data collection as recited in claim 125, wherein the data collection is implemented as a relational database.

137. A data collection as recited in claim 125, wherein the data collection is implemented as a plurality of files organized in a plurality of directories of a computer system.

138. A database for storing a plurality of data, comprising:
oligonucleotides corresponding to a plurality of known targets, or complements thereof, and
at least one score for indicating the suitability of each oligonucleotide for detecting at least one of the plurality of known targets.

139. A database as recited in claim 138, wherein the oligonucleotides are organized as sets, and further comprising at least one score for indicating the suitability of each oligonucleotide set for detecting at least one of the plurality of known targets.

140. A database as recited in claim 139, wherein each oligonucleotide set comprises at least one forward primer, at least one reverse primer, and at least one probe.

141. A database as recited in claim 140, wherein each oligonucleotide set comprises a plurality of oligonucleotides for detecting and/or amplifying a particular genomic region.

142. A computer-implemented system for identifying oligonucleotide sets for detecting target nucleic acids, comprising:
software for selecting oligonucleotides for detecting target nucleic acids;
a database for storing a plurality of data, including—
data indicative of oligonucleotide sets corresponding to a plurality of known targets, or complements thereof, and
for each target, data relating to decisions for selecting oligonucleotides for detecting the respective target,
wherein the software includes code for writing to the database data relating to decisions for selecting oligonucleotides for a particular target.

143. A computer-implemented system as recited in claim 142, wherein the software for selecting oligonucleotides includes software for performing alignments, and wherein the data relating to decisions for selecting oligonucleotides includes alignments performed by the software.


SEQUENCE LISTING (100 sequences)

SEQ ID NO: 1 - DNA, 25 bp, Artificial Sequence
  Description of Artificial Sequence Synthetic primer
ctcatggaatggctaaagacaagac

SEQ ID NO: 2 - DNA, 23 bp, Artificial Sequence
  Description of Artificial Sequence Synthetic probe
agtcctcgctcactgggcacggt

SEQ ID NO: 3 - DNA, 23 bp, Artificial Sequence
  Description of Artificial Sequence Synthetic primer
ggcattttggacaaagcgtctac

SEQ ID NO: 4 - DNA, 126 bp, Influenza A virus
ctcatggaatggctaaagacaagaccaatcctgtcacctctgactaaggggattttggggtttgtgttcacgctcaccgtgcccagtgagcgaggactgcagcgtagacgctttgtccaaaatgcc

SEQ ID NO: 5 - DNA, 26 bp, Artificial Sequence
  Description of Artificial Sequence Synthetic primer
acaagtccttatcaactctgcataga

SEQ ID NO: 6 - DNA, 34 bp, Artificial Sequence
  Description of Artificial Sequence Synthetic probe
tcagtagcaacaagtttagcaacaagccttccac

SEQ ID NO: 7 - DNA, 25 bp, Artificial Sequence
  Description of Artificial Sequence Synthetic primer
ccatcttcttcatcctccactgtaa

SEQ ID NO: 8 - DNA, 109 bp, Influenza B virus
acaagtccttatcaactctgcatagattgaatgcatatgaccagagtggaaggcttgttgctaaacttgttgctactgatgatcttacagtggaggatgaagaagatgg

SEQ ID NO: 9 - DNA, 20 bp, Artificial Sequence
  Description of Artificial Sequence Synthetic primer
agcaagcccaccacaaaaca

SEQ ID NO: 10 - DNA, 29 bp, Artificial Sequence
  Description of Artificial Sequence Synthetic probe
cgccaaaacaaaccaccaaacaaacccaa

SEQ ID NO: 11 - DNA, 23 bp, Artificial Sequence
  Description of Artificial Sequence Synthetic primer
tgcagggtacaaagttgaacact

SEQ ID NO: 12 - DNA, 91 bp, Respiratory Syncytial Virus A
agcaagcccaccacaaaacaacgccaaaacaaaccaccaaacaaacccaataatgattttcactttgaagtgttcaactttgtaccctgca

SEQ ID NO: 13 - DNA, 29 bp, Artificial Sequence
  Description of Artificial Sequence Synthetic primer
tcataattgcagccataatattcatcatc

SEQ ID NO: 14 - DNA, 34 bp, Artificial Sequence
  Description of Artificial Sequence Synthetic probe
tgccaatcacaaagttacactaacaacggtcaca

SEQ ID NO: 15 - DNA, 23 bp, Artificial Sequence
  Description of Artificial Sequence Synthetic primer
gctaaccctttctggtgagactt

SEQ ID NO: 16 - DNA, 140 bp, Respiratory Syncytial Virus B
tcataattgcagccataatattcatcatctctgccaatcacaaagttacactaacaacggtcacagttcaaacaataaaaaaccacactgaaaaaaacatcaccacttaccttactcaagtcccaccagaaagggttagc

SEQ ID NO: 17 - DNA, 29 bp, Artificial Sequence
  Description of Artificial Sequence Synthetic primer
ttttgttcattttggtatagcacaatctt

SEQ ID NO: 18 - DNA, 31 bp, Artificial Sequence
  Description of Artificial Sequence Synthetic probe
aaatcccttcaactctactgccacctctggt

SEQ ID NO: 19 - DNA, 24 bp, Artificial Sequence
  Description of Artificial Sequence Synthetic primer
cctgcaccataggcattcataaac

SEQ ID NO: 20 - DNA, 96 bp, Respiratory Syncytial Virus A
ttttgttcattttggtatagcacaatcttctaccagaggtggcagtagagttgaagggatttttgcaggattgtttatgaatgcctatggtgcagg

SEQ ID NO: 21 - DNA, 28 bp, Artificial Sequence
  Description of Artificial Sequence Synthetic primer
gaagatgcaaatcataaattcacaggat

SEQ ID NO: 22 - DNA, 37 bp, Artificial Sequence
  Description of Artificial Sequence Synthetic probe
ttcccttcctaacctggacatagcatataacatacct

SEQ ID NO: 23 - DNA, 28 bp, Artificial Sequence
  Description of Artificial Sequence Synthetic primer
actccattagctttaacatgatatccag

SEQ ID NO: 24 - DNA, 122 bp, Respiratory Syncytial Virus B
gaagatgcaaatcataaattcacaggattaataggtatgttatatgctatgtccaggttaggaagggaagacactataaagatacttaaagatgctggatatcatgttaaagctaatggagt

SEQ ID NO: 25 - DNA, 28 bp, Artificial Sequence
  Description of Artificial Sequence Synthetic primer
acgtgttaatcctaccataatgtactca

SEQ ID NO: 26 - DNA, 32 bp, Artificial Sequence
  Description of Artificial Sequence Synthetic probe
aagcagtagcccttcccgaaatgagtgataca

SEQ ID NO: 27 - DNA, 27 bp, Artificial Sequence
  Description of Artificial Sequence Synthetic primer
tattaaggctggtttggttgatttcaa

SEQ ID NO: 28 - DNA, 167 bp, Parainfluenza 1 Virus
acgtgttaatcctaccataatgtactcaaatacctcaaaaatcatcaacatgctaagactcaaaaatggacaattagaggcagcatacactactacatcatgtatcactcatttcgggaagggctactgcttccacattgttgaaatcaaccaaaccagccttaata

SEQ ID NO: 29 - DNA, 22 bp, Artificial Sequence
  Description of Artificial Sequence Synthetic primer
tcgatttgctggagcctttctc

SEQ ID NO: 30 - DNA, 31 bp, Artificial Sequence
  Description of Artificial Sequence Synthetic probe
ccaaccgaaccaatcccacattctacactgc

SEQ ID NO: 31 - DNA, 29 bp, Artificial Sequence
  Description of Artificial Sequence Synthetic primer
gatgagcccatttcaattattatcaaaca

SEQ ID NO: 32 - DNA, 198 bp, Parainfluenza 2 Virus
tcgatttgctggagcctttctcagaaatgagtccaaccgaaccaatcccacattctacactgcatcagccagcgccctactaaatactaccggattcaacaacaccaatcacaaagcagcatatacgtcttcaacctgctttaagaatactggaactcaaaagatttattgtttgataataattgaaatgggctcatc

SEQ ID NO: 33 - DNA, 25 bp, Artificial Sequence
  Description of Artificial Sequence Synthetic primer
aatggacatggcataatgtgctatc

SEQ ID NO: 34 - DNA, 34 bp, Artificial Sequence
  Description of Artificial Sequence Synthetic probe
tgagtctaatatgacagatgacacaatgctccct

SEQ ID NO: 35 - DNA, 24 bp, Artificial Sequence
  Description of Artificial Sequence Synthetic primer
gttatgactgggttcactctcgat

SEQ ID NO: 36 - DNA, 181 bp, Parainfluenza 3 Virus
aatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataac

SEQ ID NO: 37 - DNA, 23 bp, Artificial Sequence
  Description of Artificial Sequence Synthetic primer
aagactggttcctggttcagatg

SEQ ID NO: 38 - DNA, 33 bp, Artificial Sequence
  Description of Artificial Sequence Synthetic probe
aattaacctcatcaaccacctgcctgctcatag

SEQ ID NO: 39 - DNA, 22 bp, Artificial Sequence
  Description of Artificial Sequence Synthetic primer
tggtaaggtgacggctttgtag

SEQ ID NO: 40 - DNA, 173 bp, Adenovirus-B
aagactggttcctggttcagatgcttgccaattacaacattggctaccagggcttttacatccctgagggatacaaggatcgcatgtactcctttttcagaaacttccagcctatgagcaggcaggtggttgatgaggttaattacactgactacaaagccgtcaccttacca

SEQ ID NO: 41 - DNA, 22 bp, Artificial Sequence
  Description of Artificial Sequence Synthetic primer
tggtcttacatgcacatctcgg

SEQ ID NO: 42 - DNA, 24 bp, Artificial Sequence
  Description of Artificial Sequence Synthetic probe
aggacgcctcggagtacctgagcc

SEQ ID NO: 43 - DNA, 20 bp, Artificial Sequence
  Description of Artificial Sequence Synthetic primer
ctgaagtacgtctcggtggc

SEQ ID NO: 44 - DNA, 93 bp, Adenovirus-C
tggtcttacatgcacatctcgggccaggacgcctcggagtacctgagcccccgggctggtgcagtttgcccgcgccaccgagacgtacttcag

SEQ ID NO: 45 - DNA, 20 bp, Artificial Sequence
  Description of Artificial Sequence Synthetic primer
agccaacctgtggaggaact

SEQ ID NO: 46 - DNA, 32 bp, Artificial Sequence
  Description of Artificial Sequence Synthetic probe
cctctatgccaatgttgccctctatttgcctg

SEQ ID NO: 47 - DNA, 19 bp, Artificial Sequence
  Description of Artificial Sequence Synthetic primer
ttggtgggcagggtgatgt

SEQ ID NO: 48 - DNA, 94 bp, Adenovirus-E
agccaacctggaggaacttcctctatgccaatgttgccctctatttgcctgataaatacaaatacacaccggccaacatcaccctgcccaccaa

SEQ ID NO: 49 - DNA, 41 bp, Artificial Sequence
  Description of Artificial Sequence Synthetic primer
gatcttgaggctctcatggaatggctaaagacaagaccaat

SEQ ID NO: 50 - DNA, 62 bp, Artificial Sequence
  Description of Artificial Sequence Synthetic primer
tcggcattttggacaaagcgtctacgctgcagtcctcgctcactgggcacggtgagcgtgaa

SEQ ID NO: 51 - DNA, 131 bp, Artificial Sequence
  Description of Artificial Sequence Synthetic primer
aatggatacaagtccttatcaactctgcatagattgaatgcatatgaccagagtggaaggcttgttgctaaacttgttgctactgatgatcttacagtggaggatgaagaagatggccatcggatcctcaa

SEQ ID NO: 52 - DNA, 50 bp, Artificial Sequence
  Description of Artificial Sequence Synthetic primer
agcaagcccaccacaaaacaacgccaaaacaaaccaccaaacaaacccaa

SEQ ID NO: 53 - DNA, 58 bp, Artificial Sequence
  Description of Artificial Sequence Synthetic primer
gttggattgttgctgcatatgctgcagggtacaaagttgaacacttcaaagtgaaaat

SEQ ID NO: 54 - DNA, 98 bp, Artificial Sequence
  Description of Artificial Sequence Synthetic primer
ttttggcaatgataatctcaacctctctcataattgcagccataatattcatcatcatctctgccaatcacaaagttacactaacaacggtcacagtt

SEQ ID NO: 55 - DNA, 62 bp, Artificial Sequence
  Description of Artificial Sequence Synthetic primer
ggttgtttggatgggctaaccctttctggtgagacttgagtaaggtaagtggtgatgttttt

SEQ ID NO: 56 - DNA, 120 bp, Artificial Sequence
  Description of Artificial Sequence Synthetic primer
cactttatagatgtttttgttcattttggtatagcacaatcttctaccagaggtggcagtagagttgaagggatttttgcaggattgtttatgaatgcctatggtgcagggcaagtgatg

SEQ ID NO: 57 - DNA, 101 bp, Artificial Sequence
  Description of Artificial Sequence Synthetic primer
aacaaactatgtggtatgctattaatcactgaagatgcaaatcataaattcacaggattaataggtatgttatatgctatgtccaggttaggaagggaaga

SEQ ID NO: 58 - DNA, 71 bp, Artificial Sequence
  Description of Artificial Sequence Synthetic primer
ttgacgatatgttgttatatctactccattagctttaacatgatatccagcatctttaagtatctttatag

SEQ ID NO: 59 - DNA, 34 bp, Artificial Sequence
  Description of Artificial Sequence Synthetic primer
acatcacgtgttaatcctaccataatgtactcaa

SEQ ID NO: 60 - DNA, 105 bp, Artificial Sequence
  Description of Artificial Sequence Synthetic primer
acttgtcttgaacaacataggttgtaaggtattaaggctggtttggttgatttcaacaatgtggaagcagtagcccttcccgaaatgagtgatacatgatgtagt

SEQ ID NO: 61 - DNA, 30 bp, Artificial Sequence
  Description of Artificial Sequence Synthetic primer
cccaactatcgatttgctggagcctttctc

SEQ ID NO: 62 - DNA, 42 bp, Artificial Sequence
  Description of Artificial Sequence Synthetic probe
aaatgagtccaaccgaaccaatcccacattctacactgcatc

SEQ ID NO: 63 - DNA, 66 bp, Artificial Sequence
  Description of Artificial Sequence Synthetic primer
aaatggtattatttggaactcccctaaaagagatgagcccatttcaattattatcaaacaataaat

SEQ ID NO: 64 - DNA, 42 bp, Artificial Sequence
  Description of Artificial Sequence Synthetic primer
ataaaatggacatggcataatgtgctatcaagaccaggaaac

SEQ ID NO: 65 - DNA, 36 bp, Artificial Sequence
  Description of Artificial Sequence Synthetic probe
tgagtctaatatgacagatgacacaatgctccctgt

SEQ ID NO: 66 - DNA, 36 bp, Artificial Sequence
  Description of Artificial Sequence Synthetic primer
tgttgagtaagttatgactgggttcactctcgattt

SEQ ID NO: 67 - DNA, 41 bp, Artificial Sequence
  Description of Artificial Sequence Synthetic primer
aacatgaccaaagactggttcctggttcagatgcttgccaa

SEQ ID NO: 68 - DNA, 97 bp, Artificial Sequence
  Description of Artificial Sequence Synthetic primer
attggtaaggtgacggctttgtagtcagtgtaattaacctcatcaaccacctgcctgctcataggctggaagtttctgaaaaaggagtacatgcgat

SEQ ID NO: 69 - DNA, 93 bp, Artificial Sequence
  Description of Artificial Sequence Synthetic primer
atggctaccccttcgatgatgccgcagtggtcttacatgcacatctcgggccaggacgcctcggagtacctgagcccccgggctggtgcagtt

SEQ ID NO: 70 - DNA, 53 bp, Artificial Sequence
  Description of Artificial Sequence Synthetic primer
gccaccgtggggtttctaaacttgttattcaggctgaagtacgtctcggtggc

SEQ ID NO: 71 - DNA, 127 bp, Artificial Sequence
  Description of Artificial Sequence Synthetic primer
acatccaagccaacctgtggaggaacttcctctatgccaatgttgccctctatttgcctgataaatacaaatacacaccggccaacatcaccctgcccaccaacaccaacacctacgagtacatgaa

SEQ ID NO: 72 - DNA, 23 bp, Artificial Sequence
  Description of Combined DNA/RNA Molecule Synthetic probe
  Description of Artificial Sequence Synthetic probe
  modified_base (6): a, g, c, t, u or a functional equivalent
  modified_base (9): a, g, c, t, u or a functional equivalent
  modified_base (13): a, g, c, t, u or a functional equivalent
  modified_base (15): a, g, c, t, u or a functional equivalent
agtccncgntcantnggcacggt

SEQ ID NO: 73 - DNA, 25 bp, Artificial Sequence
  Description of Combined DNA/RNA Molecule Synthetic primer
  Description of Artificial Sequence Synthetic primer
  modified_base (5): a, g, c, t, u or a functional equivalent
  modified_base (9): a, g, c, t, u or a functional equivalent
  modified_base (18): a, g, c, t, u or a functional equivalent
  modified_base (21): a, g, c, t, u or a functional equivalent
  modified_base (23): a, g, c, t, u or a functional equivalent
ctcanggantggctaaanacnanac

SEQ ID NO: 74 - DNA, 23 bp, Artificial Sequence
  Description of Combined DNA/RNA Molecule Synthetic primer
  Description of Artificial Sequence Synthetic primer
  modified_base (1): a, g, c, t, u or a functional equivalent
  modified_base (4)..(5): a, g, c, t, u or a functional equivalent
  modified_base (7): a, g, c, t, u or a functional equivalent
  modified_base (10): a, g, c, t, u or a functional equivalent
  modified_base (16): a, g, c, t, u or a functional equivalent
  modified_base (20): a, g, c, t, u or a functional equivalent
ngcnntntgnacaaancgtntac

SEQ ID NO: 75 - DNA, 34 bp, Artificial Sequence
  Description of Combined DNA/RNA Molecule Synthetic probe
  Description of Artificial Sequence Synthetic probe
  modified_base (24): a, g, c, t, u or a functional equivalent
  modified_base (27)..(30): a, g, c, t, u or a functional equivalent
  modified_base (34): a, g, c, t, u or a functional equivalent
tcagtagcaacaagtttagcaacnagnnnnccan

SEQ ID NO: 76 - DNA, 26 bp, Artificial Sequence
  Description of Combined DNA/RNA Molecule Synthetic primer
  Description of Artificial Sequence Synthetic primer
  modified_base (2): a, g, c, t, u or a functional equivalent
  modified_base (6): a, g, c, t, u or a functional equivalent
  modified_base (8): a, g, c, t, u or a functional equivalent
  modified_base (11): a, g, c, t, u or a functional equivalent
  modified_base (13): a, g, c, t, u or a functional equivalent
  modified_base (20)..(21): a, g, c, t, u or a functional equivalent
anaagncnttntnaactctnnataga

SEQ ID NO: 77 - DNA, 25 bp, Artificial Sequence
  Description of Combined DNA/RNA Molecule Synthetic primer
  Description of Artificial Sequence Synthetic primer
  modified_base (8): a, g, c, t, u or a functional equivalent
  modified_base (24)..(25): a, g, c, t, u or a functional equivalent
ccatcttnttcatcctccactgtnn

SEQ ID NO: 78 - DNA, 31 bp, Artificial Sequence
  Description of Combined DNA/RNA Molecule Synthetic probe
  Description of Artificial Sequence Synthetic probe
  modified_base (8): a, g, c, t, u or a functional equivalent
  modified_base (17): a, g, c, t, u or a functional equivalent
aaatcccntcaactctnctgccacctctggt

SEQ ID NO: 79 - DNA, 24 bp, Artificial Sequence
  Description of Combined DNA/RNA Molecule Synthetic primer
  Description of Artificial Sequence Synthetic primer
  modified_base (21): a, g, c, t, u or a functional equivalent
cctgcaccataggcattcatnaac

SEQ ID NO: 80 - DNA, 28 bp, Artificial Sequence
  Description of Combined DNA/RNA Molecule Synthetic primer
  Description of Artificial Sequence Synthetic primer
  modified_base (8): a, g, c, t, u or a functional equivalent
gaagatgnaaatcataaattcacaggat

SEQ ID NO: 81 - DNA, 32 bp, Artificial Sequence
  Description of Combined DNA/RNA Molecule Synthetic probe
  Description of Artificial Sequence Synthetic probe
  modified_base (12): a, g, c, t, u or a functional equivalent
  modified_base (15): a, g, c, t, u or a functional equivalent
  modified_base (18): a, g, c, t, u or a functional equivalent
  modified_base (20): a, g, c, t, u or a functional equivalent
  modified_base (27): a, g, c, t, u or a functional equivalent
aagcagtagccnttnccnanatgagtnataca

SEQ ID NO: 82 - DNA, 28 bp, Artificial Sequence
  Description of Combined DNA/RNA Molecule Synthetic primer
  Description of Artificial Sequence Synthetic primer
  modified_base (8)..(9): a, g, c, t, u or a functional equivalent
  modified_base (13): a, g, c, t, u or a functional equivalent
  modified_base (16): a, g, c, t, u or a functional equivalent
acgtgtanntccnacnataatgtactca

SEQ ID NO: 83 - DNA, 27 bp, Artificial Sequence
  Description of Combined DNA/RNA Molecule Synthetic primer
  Description of Artificial Sequence Synthetic primer
  modified_base (4): a, g, c, t, u or a functional equivalent
  modified_base (13): a, g, c, t, u or a functional equivalent
tatnaaggctggnttggttgatttcaa

SEQ ID NO: 84 - DNA, 31 bp, Artificial Sequence
  Description of Combined DNA/RNA Molecule Synthetic probe
  Description of Artificial Sequence Synthetic probe
  modified_base (11): a, g, c, t, u or a functional equivalent
  modified_base (23): a, g, c, t, u or a functional equivalent
ccaaccgaacnaatcccacattntacactgc

SEQ ID NO: 85 - DNA, 22 bp, Artificial Sequence
  Description of Combined DNA/RNA Molecule Synthetic primer
  Description of Artificial Sequence Synthetic primer
  modified_base (22): a, g, c, t, u or a functional equivalent
tcgatttgctggagcctttctn

SEQ ID NO: 86 - DNA, 29 bp, Artificial Sequence
  Description of Combined DNA/RNA Molecule Synthetic primer
  Description of Artificial Sequence Synthetic primer
  modified_base (15): a, g, c, t, u or a functional equivalent
  modified_base (24): a, g, c, t, u or a functional equivalent
gatgagcccatttcnattattatnaaaca

SEQ ID NO: 87 - DNA, 34 bp, Artificial Sequence
  Description of Combined DNA/RNA Molecule Synthetic probe
  Description of Artificial Sequence Synthetic probe
  modified_base (1): a, g, c, t, u or a functional equivalent
  modified_base (4): a, g, c, t, u or a functional equivalent
  modified_base (9): a, g, c, t, u or a functional equivalent
  modified_base (30)..(31): a, g, c, t, u or a functional equivalent
ngantctantatgacagatgacacaatgcnncct

SEQ ID NO: 88 - DNA, 25 bp, Artificial Sequence
  Description of Combined DNA/RNA Molecule Synthetic primer
  Description of Artificial Sequence Synthetic primer
  modified_base (17): a, g, c, t, u or a functional equivalent
  modified_base (21): a, g, c, t, u or a functional equivalent
aatggacatggcataangtgntatc

SEQ ID NO: 89 - DNA, 24 bp, Artificial Sequence
  Description of Combined DNA/RNA Molecule Synthetic primer
  Description of Artificial Sequence Synthetic primer
  modified_base (15): a, g, c, t, u or a functional equivalent
  modified_base (23): a, g, c, t, u or a functional equivalent
gttatgactgggttnactctcgnt

SEQ ID NO: 90 - DNA, 24 bp, Artificial Sequence
  Description of Combined DNA/RNA Molecule Synthetic probe
  Description of Artificial Sequence Synthetic probe
  modified_base (17): a, g, c, t, u or a functional equivalent
  modified_base (20): a, g, c, t, u or a functional equivalent
aggacgcctcggagtanctnagcc

SEQ ID NO: 91 - DNA, 22 bp, Artificial Sequence
  Description of Combined DNA/RNA Molecule Synthetic primer
  Description of Artificial Sequence Synthetic primer
  modified_base (5): a, g, c, t, u or a functional equivalent
tggtnttacatgcacatctcgg

SEQ ID NO: 92 - DNA, 32 bp, Artificial Sequence
  Description of Combined DNA/RNA Molecule Synthetic probe
  Description of Artificial Sequence Synthetic probe
  modified_base (4): a, g, c, t, u or a functional equivalent
  modified_base (16): a, g, c, t, u or a functional equivalent
  modified_base (18): a, g, c, t, u or a functional equivalent
cctntatgccaatgtngncctctatttgcctg

SEQ ID NO: 93 - DNA, 20 bp, Artificial Sequence
  Description of Combined DNA/RNA Molecule Synthetic primer
  Description of Artificial Sequence Synthetic primer
  modified_base (16): a, g, c, t, u or a functional equivalent
agccaacctgtggagnaact

SEQ ID NO: 94 - DNA, 19 bp, Artificial Sequence
  Description of Combined DNA/RNA Molecule Synthetic primer
  Description of Artificial Sequence Synthetic primer
  modified_base (5): a, g, c, t, u or a functional equivalent
ttggngggcagggtgatgt

SEQ ID NO: 95 - DNA, 23 bp, Artificial Sequence
  Description of Artificial Sequence Synthetic primer
cttctaaccgaggtcgaaacgta

SEQ ID NO: 96 - DNA, 25 bp, Artificial Sequence
  Description of Artificial Sequence Synthetic primer
cgtctacgctgcagtcctcgctcac

SEQ ID NO: 97 - DNA, 37 bp, Artificial Sequence
  Description of Artificial Sequence Synthetic probe
ggctaaagacaagaccaatcctgtcacctctgactaa

SEQ ID NO: 98 - DNA, 10 bp, Artificial Sequence
  Description of Artificial Sequence Synthetic oligonucleotide
aaacacgtgc

SEQ ID NO: 99 - DNA, 10 bp, Artificial Sequence
  Description of Artificial Sequence Synthetic oligonucleotide
ccttgttcca

SEQ ID NO: 100 - DNA, 10 bp, Artificial Sequence
  Description of Artificial Sequence Synthetic oligonucleotide
cagggacgat